(19) United States
(12) Patent Application Publication
(10) Pub. No.: US 2018/0366221 A1
(43) Pub. Date: Dec. 20, 2018

(54) PLATFORM FOR CONTEXT BASED SYNDROMIC SURVEILLANCE

(71) Applicant: RADICALOGIC TECHNOLOGIES INC. DBA RL SOLUTIONS, Toronto (CA)

(72) Inventors: Yves CREHORE, Toronto (CA); Tom BELCHER, Toronto (CA); Jason LLORIN, Toronto (CA)

(21) Appl. No.: 16/007,839

(22) Filed: Jun. 13, 2018

Related U.S. Application Data
(60) Provisional application No. 62/573,008, filed on Oct. 16, 2017.
(60) Provisional application No. 62/518,816, filed on Jun. 13, 2017.

(51) Int. Cl. G16H 40/60; G16H 50/30; G16H 70/00; G06F 17/30

(52) U.S. Cl. CPC G16H 40/60; G06F 17/30292; G16H 70/00; G16H 50/30

(57) ABSTRACT

Embodiments described herein relates to a platform for infection surveillance that can assist infection preventionists and antimicrobial stewardship teams with infection detection rules and dynamic alerts by aggregating and processing infection surveillance data with contextual healthcare data regarding a facility, organization, and so on. The platform can enable proactive prevention of infection risks by detecting risk patterns in the data and generating alerts.

### CROSS REFERENCE

This application claims all benefit, including priority, of U.S. Application No. 62/573,008, filed 16 Oct. 2017, entitled “PLATFORM FOR CONTEXT BASED SYNDROMIC SURVEILLANCE”, and U.S. Application No. 62/518,816, filed 13 Jun. 2017, entitled “PLATFORM FOR CONTEXT BASED SYNDROMIC SURVEILLANCE”, all of which are hereby incorporated by reference.

### FIELD

The improvements generally relate to the field of healthcare systems and more specifically to improved devices, computer implemented methods, and computer readable media directed to infection surveillance.

### INTRODUCTION

Each year, forecasts are distributed by central reporting agencies to healthcare institutions, providing information about various illnesses, such as influenza for example, and what can be expected for an upcoming season, including estimates about onset, duration and severity. A healthcare system can manage important health care data for different types of heath care facilities.

The ability to monitor and track infections is also becoming a burden. According to the Hospital Association of Southern California, routine surveillance now takes up roughly 25-30% of an infection preventionists (IP)'s time. Freeing up IPs' time to handle the growing list of infection prevention tasks is critical to the successful operation of any healthcare organization: outbreak investigations, epidemiologic investigations, prevention measures, disaster planning and even bioterrorism.

### SUMMARY

Surveillance of infections is a technically challenging endeavor, as myriad dynamic data sets are continuously received and it is difficult for a system to interpret. To reduce the burden on IPs, while at the same time improving infection surveillance capabilities, many healthcare organizations have started using infection prevention and control software (IPCS). Effective infection control software does not require data re-entry and leads to better detection, management, prevention and control of infection risks.

In the healthcare industry, an infection tracking system is invaluable for IPs as time is of the essence in taking action to mitigate further spread of an infection and to address root causes. A quick response can save lives, and responses include interventions such as establishing quarantines, increasing hygiene standards, segregating populations, conducting fascinations, prescribing pre-emptive medication, among others. The infection tracking system needs to be flexibly adaptable to receiving contextual information, and this is a major technical challenge due to the inflexibility of existing healthcare information systems which all must interact and interoperate with one another. The infection tracking system, of some embodiments described herein, utilizes an innovative meta-base data structure backend to avoid requiring any retooling of existing healthcare information systems that are present in the context of a facility or a group of facilities.

Accordingly, an improved electronic device configured for monitoring a population or sub-population of people in respect of syndromic characteristics of a potential infection event or infection vector is described in various embodiments. The electronic device, in some embodiments, is an improved computer system that is adapted for (1) extending a flexible storage backend (e.g., a metabase) to capture information associated with syndromic surveillance without requiring modifications to applications that interface with the storage backend, and/or (2) rendering an improved infection charting interface that graphs events and renders trend lines associated thereof.

One or a combination of these features are provided. For example, in a first preferred embodiment, an improved database and data structure backend is provided. A computer system is provided for dynamically incorporating syndromic surveillance data objects in a backend data structure having a flexible meta-schema allowing a new or modified taxonomy to be adopted or accommodated without modifications to applications that interconnect with the backend data structure.

The system includes a user interface component on a connected device configured to receive a request to create a new syndromic surveillance data object at a coordinate position of a graphical interface being rendered on a display, the new syndromic surveillance data object including at least a field name and a data value extracted from a corresponding point along an axis of a graph being rendered on the display; a storage device having a metabase associated therewith to maintain the meta-schema; and at least one processor configured to execute instructions to provide an object insertion engine and a system interface.

The object insertion engine is configured to establish a communication link between the system interface and the user interface component over a network, receive, at the system interface from the user interface component via the network, the request to generate the new syndromic surveillance data object using the object insertion engine, the new syndromic surveillance data object representative of an event that is being tracked to identify potential patterns between the event as represented by new syndromic surveillance data object and other field objects maintained on the metabase, store in the storage device, a dictionary of field objects in the metabase, wherein an instance of a field object is a form field, and wherein the form field is configured to receive a field value, the metabase structuring the dictionary of field objects into corresponding meta-tables and establishing meta-relations defining parent-child relationships between nodes representing the one or more field objects of the metabase, the meta-relations being maintained as corresponding foreign keys used to reference the corresponding field objects and the corresponding meta-tables, the metabase configured to have a dynamic number of columns that is dynamic according to a number of field objects in the dictionary, process the request from the user interface component to add the new syndromic surveillance data object in the metabase, and automatically maintain, by the metabase, electronic representations of relationships between the field objects and the form fields, the one or more additional user-defined field objects representing the new or modified taxonomy.

The object insertion engine is configured to automatically maintain the electronic representations by: establishing, by the metabase, one or more new columns corresponding to the new syndromic surveillance data object; and establishing new meta-relations defining the one or more additional user-defined field objects as new child nodes to corresponding parent nodes of the one or more additional user-defined field objects.

The one or more new columns, the meta-tables, and the new meta-relations defining the one or more additional user-defined field objects as the new child nodes in concert represent the new or modified taxonomy incorporating the inserted new syndromic surveillance data object. Foreign keys are made accessible for referencing, by the applications that interconnect with the backend data structure, the one or more new columns associated with the new syndromic surveillance data object.

In another aspect, the new graphical syndromic surveillance data object includes at least a syndromic originator spatial coordinate, and wherein during the establishing of the one or more new columns corresponding to the new graphical syndromic surveillance data object, at least one of the one or more new columns is populated with a set of field value representing a spatial coordinate distance between a healthcare facility associated with a data record and the syndromic originator spatial coordinate. In another aspect, each spatial coordinate distance is weighted by a corresponding transmission speed factor established between the healthcare facility and the event.

In another aspect, the data value extracted from the corresponding point along an axis of a graph being rendered on the display is representative of an approximate point in time in which the event occurred. In another aspect, the data value is temporally shifted based on a time-lag factor. In another aspect, the time-lag factor is determined based at least in reference to a lookup table associating a type of infection associated with the event and an incubation period associated with the type of infection.

In another aspect, the new graphical syndromic surveillance data object includes at least a syndromic originator temporal coordinate, and wherein during the establishing of the one or more new columns corresponding to the new graphical syndromic surveillance data object, at least one of the one or more new columns are populated with a field value representing a temporal distance between healthcare record time stamp entry associated with a data record and the syndromic originator temporal coordinate.

In another aspect, the one or more new columns corresponding to the new syndromic surveillance data object includes a binomial value indicative of whether the event has affected the underlying values stored in the field object.

In another aspect, at least one processor is further configured to execute instructions to provide a graphical display rendering engine that is configured to render a graphical display indicative of one or more records stored in the metabase, the graphical display rendering at least (i) a graph plotting a number of events that match an infection criteria during a duration of time and (ii) a trend line plotting a statistical average of the number of events that match an infection criteria during the duration of time.

In another aspect, the metabase is queried to modify a visual characteristic associated with the plotting of the one or more records whose corresponding binomial values of the one or more new columns indicate that the event has affected the underlying values stored in the field object.

In a second preferred embodiment, an improved decision support interface system for infection prevention is provided. The improved electronic device is adapted to interoperate with a display of a computing device, generating instruction sets for rendering an interface for display that presents visual, symbolic representations corresponding to specific events as extracted from data sets. A user input is received at it input receiver coupled to the computing device representing selections of the visual symbolic representations presented to the person through the display screen.

The electronic device is a computer system that is configured for dynamically rendering syndromic surveillance data objects as interactive graphical visual elements on the display screen. The syndromic surveillance data objects each represent a corresponding event that is being tracked to identify one or more patterns in relation to one or more underlying variables extracted from a backend data structure storing one or more electronic health records.

The computer system includes a data receiver configured to extract from the backend data structure one or more data sets representing healthcare incident records that match one or more logical conditions indicative of a syndromic surveillance case definition; a graphics rendering engine configured to render a graph along a dimensional axis having a plurality of points each indicative of a number of the healthcare incident records that match the syndromic surveillance case definition at a corresponding value along the dimensional axis, and configured to render one or more interactive graphical event markers visually proximate to the graph, the interactive graphical event markers each generated responsive to a detected user input; and a trend line determination engine configured to transform the plurality of points into a trend line along the dimensional axis by applying one or more curve fitting techniques to smooth the trend line and to render the trend line as an graphical overlay overlaid in respect of the graph.

In another aspect, the graph and the trend line are rendered using the d3™ data visualization tool set, which is more suited for drawing the whole graph, and the one or more interactive graphical event markers are rendered using a React framework which is more suited for creating isolated declarative components, to avoid re-rendering whenever an interaction occurs in relation to a graphical event marker.

In another aspect, the dimensional axis is time, and wherein the trend line includes a forecasting segment that extends beyond a present time, the forecasting segment determined based on an extrapolation of the trend line.

In another aspect, the forecasting segment is modified based on a presence of an intervention event as designated by at least one of the interactive graphical event markers, and the system further comprises a pattern recognition engine configured to determine a correlation level between a classification of the intervention event and the syndromic surveillance case definition, the correlation level utilized by the trend line determination engine to bias the extrapolation of the trend line, modifying the rendering of the forecasting segment.

In another aspect, the one or more curve fitting techniques includes at least one of a rolling average, Holt Winters curve fitting, and polynomial regression.

In another aspect, the data receiver is configured to interconnect with one or more healthcare facilities or one or more segments of healthcare facilities, and the one or more data sets include an approximate location distance value for each of the healthcare incident records determined based on an approximate location value for the corresponding healthcare incident record compared against an syndromic originator spatial coordinate representative of a location of a known syndromic event; and the graphics rendering engine is configured to render an interactive interface component, which when interfaced with, cause the graph and the trend line to be re-rendered absent points representing healthcare incident records having a sufficient proximity to the known syndromic event based on the approximate location distance value being below a pre-defined threshold.

In a third embodiment, the improved database and data structure backend is combined with the improved decision support interface system for infection prevention such that new objects created on the decision support interface system cause insertions in the database and data structure backend (e.g., schema changes), while maintaining interoperability with other systems that interact with the database and data structure backend (e.g., an adverse events reporting system that is designed to operate with a static schema). Improvements to a hospital information system are thus described.

In another aspect, the backend data structure includes a flexible meta-schema allowing a new or modified taxonomy to be adopted or accommodated without modifications to applications that interconnect with the backend data structure.

In another aspect, the backend data structure includes a flexible meta-schema allowing a new or modified taxonomy to be adopted or accommodated without modifications to applications that interconnect with the backend data structure and the system further comprises a storage device having a metabase associated therewith to maintain the meta-schema.

In another aspect, the metabase includes a dictionary of field objects in the metabase, wherein an instance of a field object is a form field, and wherein the form field is configured to receive a field value, the metabase structuring the dictionary of field objects into corresponding meta-tables and establishing meta-relations defining parent-child relationships between nodes representing the one or more field objects of the metabase, the meta-relations being maintained as corresponding foreign keys used to reference the corresponding field objects and the corresponding meta-tables, the metabase configured to have a dynamic number of columns that is dynamic according to a number of field objects in the dictionary; and the new syndromic surveillance data objects are incorporated into the metabase in one or more new columns corresponding to each new syndromic surveillance data object, establishing new meta-relations defining the one or more additional user-defined field objects as new child nodes to corresponding parent nodes of the one or more additional user-defined field objects. At least one of the one or more new columns is populated with a set of field value representing the approximate location distance value.

The electronic device generates a visual representation of syndromic surveillance data. The visual representation, in a preferred embodiment, is a graph or, in another embodiment, is a bar chart. The visual representation of the data is provided to indicate a specific type of syndrome, such as a failed febrile respiratory illness over time, based on data feeds received from one or more healthcare facilities or practitioners.

A trend line is mapped across a duration of time and rendered on the display that smooths out fluctuations in data to show a pattern or trend of data more clearly. The trend line, for example, can include a moving average trend line wherein a number of points in a period are averaged, and the average value is utilized to determine points along the trend line. The trend line is useful to indicate gradual shifts in an average, a user would be able to visually inspect how the trend shifts over time. A visual inspection of the actual data against the trend line is also useful to indicate the underlying data that is causing the trend line to increase or decrease over time, as events outside of the average range occur. The trend line is especially useful in observing durations where periodicity and other types of patterns are observable in the underlying data.

One or more event makers are established through the input receiver from the user, by way of a sentinel event signal. The sentinel events are triggered when the underlying logic of the sentinel event is satisfied.

One or more intervention event pointers are established through the input receiver by the user, by way of an intervention event signal. The intervention event is indicative of a point in time in which an intervention is known to have taken place. The intervention event can include, for example, adoption of best practices, an increased infection protocol, among others. Similarly, an adoption period duration may be associated with the intervention event pointer such that delayed effects of the intervention may be observed.

In accordance with an aspect, there is provided a healthcare system with a processor and persistent data store with instructions to configure the processor to generate alerts by processing infection surveillance data and health care data for a particular context, such as a health care facility, organization or region.

In accordance with an aspect, there is provided a healthcare system with an interface comprising form fields and visual elements to receive infection surveillance and feedback data for a persistent data store.

In accordance with an aspect, there is provided a healthcare system comprising a processor and persistent data store with instructions to configure the processor to provide: an interface comprising form fields and visual elements to receive health infection care and feedback data for a persistent data store and display infection alerts; a data management component to: receive infection surveillance data feeds; process the infection surveillance data feeds along with patient, health care and feedback data to detect patterns for alerts; and store health care record entries for the health care and feedback data in the persistent data store; a rules engine to identify a set of rules stored in the persistent data store, each rule having a trigger based on a pattern and an action for an infection related alert, the trigger relating to the healthcare data in the persistent data store and the action relating to the visual elements; the form engine to update the form interface to display the visual elements to receive additional infection and feedback data.

In accordance with an aspect, there is provided a computer-implemented method for dynamically generating an electronic form for receiving infection data relating at a user interface component, the electronic form adapted to provide a flexible meta-schema allowing a new or modified taxonomy to be adopted or accommodated without modifications to applications that process the electronic form, the method comprising: providing a healthcare infection management system including a storage device and a processor, the storage device having a metabase associated therewith to maintain the meta-schema and the processor configured to provide a form engine and a system interface; establishing a communication link between the system interface and the user interface component over a network; receiving, at the system interface from the user interface component via the network, a request to generate the electronic form using the form engine; providing a dictionary of field objects in the metabase associated with the storage device of the healthcare infection management system, wherein an instance of a field object is a form field, and wherein the form field is configured to receive a field value, the metabase structuring the dictionary of field objects into corresponding meta-tables and establishing meta-relations defining parent-child relationships between nodes representing the one or more field objects of the metabase, the meta-relations being maintained as corresponding foreign keys used to reference the corresponding field objects and the corresponding meta-tables, the metabase configured to have a dynamic number of columns that is dynamic according to a number of field objects in the dictionary; adding one or more additional user-defined field objects in the metabase; automatically maintaining, by the metabase, electronic representations of relationships between the field objects and the form fields, the one or more additional user-defined field objects representing the new or modified taxonomy, the automatic maintaining including: establishing, by the metabase, one or more new columns corresponding to the one or more additional user-defined field objects; and establishing new meta-relations defining the one or more additional user-defined field objects as new child nodes to corresponding parent nodes of the one or more additional user-defined field objects; generating the electronic form, using the form engine configured by the processor of the healthcare incident management system, wherein the electronic form includes an ordered collection of form fields instantiated using the field objects, the ordered collection based at least on the relationships stored in the metabase including at least the meta-tables, the meta-relations, and the new meta-relations, the ordered collection of form fields being a set of visual elements; controlling the display to automatically return and display, from the system interface to the user interface component via the network, the set of visual elements, wherein a visual element corresponds to an infection alert based on patterns detected in infection surveillance data feeds; defining, on the storage device using the metabase of the healthcare infection management system; receiving, at the system interface, a selected visual element from the user interface component via the network, the selected visual element being from the set of visual elements and triggering an additional alert; controlling the electronic form using the set of visual elements based at least on the relationships stored in the metabase to adopt or accommodate the new or modified taxonomy.

Many further features and combinations thereof concerning embodiments described herein will appear to those skilled in the art following a reading of the instant disclosure.

### DESCRIPTION OF THE FIGURES

Embodiments will now be described, by way of example only, with reference to the attached figures, wherein in the figures:

FIG. 1 is a schematic of an example healthcare system according to some embodiments;

FIG. 2 is a schematic of an example healthcare system according to some embodiments;

FIG. 3 is an example alert interface with different visual elements for infection surveillance and alerts according to some embodiments;

FIG. 4 is an example alert interface with different visual elements for failed respiratory illnesses over time according to some embodiments;

FIG. 5 is an example alert interface with different visual elements to trigger display of failed respiratory illnesses with benchmarking according to some embodiments;

FIG. 6 is an example alert interface with control indicia for different visual elements according to some embodiments;

FIG. 7 is an example alert interface with control indicia for different time periods according to some embodiments;

FIG. 8 is an example alert interface with control indicia for different graph displays according to some embodiments;

FIG. 9 is an example alert interface with visual elements for different event displays according to some embodiments;

FIG. 10 is an example alert interface with visual elements for different visibility displays according to some embodiments;

FIG. 11 is an example alert interface with visual elements for different interventions according to some embodiments;

FIG. 12 is an example alert interface with visual elements for interventions over different time periods according to some embodiments;

FIG. 13 is an example alert interface with visual elements for a checklist of interventions including cleaning and disinfecting according to some embodiments;

FIG. 14 is an example alert interface with visual elements for a checklist of interventions including ISO flag types according to some embodiments;

FIG. 15 is an example alert interface with visual elements for interventions including follow-ups according to some embodiments;

FIG. 16 is an example alert interface with visual elements for intervention reports according to some embodiments;

FIG. 17 is an example alert interface with visual elements for an intervention report according to some embodiments;

FIG. 18 is an example alert interface with visual elements for a failed patient list according to some embodiments;

FIG. 19 is an example alert interface with visual elements for triggering generation of different reports according to some embodiments;

FIG. 20 is an example process for infection surveillance and features according to some embodiments;

FIG. 21 is an example process for infection surveillance according to some embodiments;

FIG. 22 is an example interface with different control indicia for report generation;

FIG. 23 depicts an example of travel across the world and example centres of interest;

FIG. 24 depicts twins of infectious disease;

FIG. 25 depicts an example for Carbapenem-resistant Enterobacteriaceae (CRE);

FIG. 26 is an example global map depicting rapid emergence of multidrug-resistant clinical Candida auris strains;

FIG. 27 is an example flow diagram of the development of multi-drug resistant parasites;

FIG. 28 describes infection risk management;

FIG. 29 is an example Context Based Syndromic Surveillance (CBSS) system according to some embodiments that includes Interface Prediction Platform;

FIG. 30 is a view of an example infection prediction platform and storage device according to some embodiments;

FIG. 31 illustrates an embodiment interface for an Infection prediction platform;

FIG. 32 is a view of an example visual interface element or display generated at infection prediction platform according to some embodiments;

FIG. 33 is a view of an example visual interface element or display generated at Infection prediction platform according to some embodiments;

FIG. 34 is a view of an example visual interface element or display generated at Infection prediction platform according to some embodiments;

FIG. 35 is a diagram of example risks and mitigation strategies that can be implemented by the system;

FIG. 36 is a diagram of example context that can be served by the system; and

FIG. 37A and FIG. 37B illustrate an example flexible metabase data structure and schema model, according to some embodiments.

### DETAILED DESCRIPTION

Tracking context is important in syndromic surveillance. Syndromic surveillance provides an infection prevention tool that helps risk management at one or more healthcare facilities. Healthcare information is continuously and systematically collected, analysed, and interpreted over a period of time to identify a threshold of early symptomatic cases. If the pattern can be detected or abnormal results indicated, these may be representative of occurrences that act as “sentinels” for early investigation and intervention.

Surveillance of this infection is a technically challenging endeavor, as myriad dynamic data sets are continuously received and it is difficult for a system to interpret. Without extensive testing, it is often difficult to diagnose a root cause (e.g., is it stomach flu or is it something more serious, such as Lyme disease) of an illness, so it is difficult to flag and identify patients.

To reduce the burden on IPs, while at the same time improving infection surveillance capabilities, many healthcare organizations have started using infection prevention and control software (IPCS). Effective infection control software does not require data re-entry and leads to better detection, management, prevention and control of infection risks.

Time is of the essence. A quick, accurate, and responsive interface is needed to provide practitioners with useful tools to enable decision making. Electronic health records and logged medical events are useful, but healthcare information infrastructure and data architecture is often dated, as a mix of newer applications and legacy applications often co-exist at healthcare data centers.

These applications are hosted on enterprise-level software and hardware, and often have heightened privacy, reliability, redundancy, and information security protections. Retooling these applications is time consuming and requires significant resources in testing and re-development, especially older applications that may be written in older computer programming languages.

Accordingly, to overcome significant compatibility issues inherent in healthcare data systems, many organizations have undertaken expensive customizations of schemas, data structures, and communication protocols utilized by the applications. Many applications therefore require pre-defined schemas and customization at the time of integration into the healthcare information system based on the specific, non-standard schema utilized by the healthcare facility. Existing standards, such as HL7, include some standardization but can be varied at each healthcare facility, leading to inconsistencies between facilities.

Reports can be used prevent outbreaks from spreading but it is hard to know the moment where efforts will have the greatest impact. Waiting for the laboratory or public health agency to confirm an outbreak can be too late.

In an example scenario, there is a 400 bed acute care community hospital at Mercy Oaks Memorial. The emergency room (ER) team reports approximately 23 patients have been seen in the in the past 24 hrs who failed the Febrile Respiratory Illness (FRI) screening. Total ER attendees for the same 24 hrs were 131 (higher than normal). IPs have followed up with occupational Health team and 9 staff members have been absent due to flu-like symptoms at Mercy Oaks Memorial over the past 2 weeks. Two staff members were hospitalized with pneumonia. Administrators at Mercy Oaks Memorial need to be able to quickly and accurately assess the infection situation.

A responsive, dynamic decision support interface is thus desirable to track infection symptoms and to manage risk by generating alerts proactively (e.g., in view of logical conditions being satisfied), track the impact of events (given their locality and time), and forecast risk. This decision support interface, in accordance with various embodiments, is configured to interoperate with an improved backend metabase which flexibly modifies an underlying data structure schema by adding new columns responsive to events being tracked. This flexible schema allows easier inter-healthcare facility (especially those that have different schemas) data collection and comparison.

Further, the flexible schema described herein built using the metaschema functionality of the metabase allows the backend data storage to expand in a memory efficient manner (e.g., expanding as necessary). In further embodiments, a memory de-allocation mechanism (e.g., a daemon process) is periodically or continually run to shrink the schema after events are no longer being tracked so that the metabase retains its effectiveness as a storage mechanism. Accordingly, as the administrator marks events for review or consideration on the graphical user interface (e.g., using a mouse input), the backend data storage automatically right sizes a number of columns based on a number of events currently being marked for review or consideration. The efficient tracking of a number of columns in the data storage aids in maintaining sufficient responsiveness of the system.

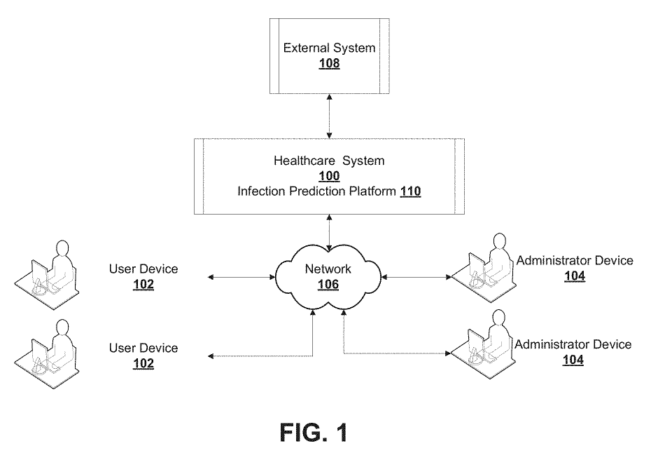

FIG. 1 is a schematic of an example healthcare system 100 according to some embodiments. The healthcare system 100 connects to external systems 108, user device 102, and administrator device 104 to receive data feeds of infection data and other healthcare related data. The healthcare system 100 uses infection prediction platform 110 to process the data and generate infection surveillance data, including alert data and infection prediction data.

The healthcare system 100 is an improved electronic device configured for monitoring a population or sub-population of people in respect of syndromic characteristics of a potential infection event or infection vector. Infection events or infection vectors include outbreaks of bacteria, viruses, fungi, as observed through continuous detection of symptoms as captured through electronic health record data. For example, people as they visit clinics and hospitals provide data sets of information which are then processed to extract relevant data for insertion into a data backend.

The infection prediction platform 110 includes infection surveillance software and/or hardware and is configured assist infection preventionists (IPs) and antimicrobial stewardship teams with access to software tools to move from reactive to proactive prevention of infection risks. The infection prediction platform 110 gives healthcare organizations meaningful, actionable data that they can use to prevent healthcare-acquired infections (HAIs) and ensure the right drugs are reaching the right patients—at the right time and for the right duration. By sharing this information across the organization using healthcare system 100, facilities can work together to make better decisions that improve patient outcomes.

The infection prediction platform 110 implements context-based syndromic surveillance (CBSS) that helps identify early indications of community disease clusters. Accordingly, infection prediction platform 110 can provide a CBSS tool for the continuous, systematic collection, analysis and interpretation of health-related data specific to a health care facility and the population presenting there. The infection prediction platform 110 can be used to identify the changing community bioburden presenting to a healthcare provider via now casting and forecasting data that precede diagnosis and signal a sufficient probability of an increasing bioburden or an outbreak to warrant further responses to mitigate facility functionality, staffing needs, materials asset allocation and specific communicable disease mitigation interventions to protect community, staff and in-patient populations.

In particular, infection prediction platform 110 is an improved computer system is provided that is adapted for (1) extending a flexible storage backend (a metabase) to capture information associated with syndromic surveillance without requiring modifications to applications that interface with the storage backend, and/or (2) rendering an improved infection charting interface that graphs events and renders trend lines associated thereof.

The infection prediction platform 110 is utilized to maintain a data structure backend storing healthcare event data records, which are flexibly maintained as new events take place. Information associated with events are utilized to provide context to renderings generated by infection prediction platform 110, or in respect of actions triggered or otherwise initiated by infection prediction platform 110. For example, reports may be generated that may also include command and control signals that cause various actions to be taken.

The infection prediction platform 110 applies syndromic surveillance to a facility's historical data and specific patient population. This helps teams intervene earlier to better protect staff and patients. With an indication of an increasing bioburden, infection teams can configure The infection prediction platform 110 uses an administrator unit 104 to specify interventions relevant to a particular estimated type of communicable disease to protect a community, staff and in-patient populations.

Syndromic surveillance is the continuous, systematic collection, analysis and interpretation of health-related data for the planning, implementation, and evaluation of public health practice. Syndromic surveillance may be used to serve as an early warning system for impending public health emergencies; document the impact of an intervention, or track progress towards specified goals; and monitor and clarify the epidemiology of health problems, allowing priorities to be set and to inform public health policy and strategies. CBSS is directed to continuous, systematic collection, analysis and interpretation of health-related data specific to a health care facility or facilities and the population presenting there. CBSS can be used to identify the changing community bioburden presenting to a healthcare provider via “now or real-time” casting and forecasting processes that precede diagnosis and signal a sufficient probability of an increasing bioburden or an outbreak to warrant further responses to mitigate facility functionality, staffing needs, materials asset allocation and specific communicable disease mitigation interventions to protect community, staff and in-patient populations.

Identifying early indicators of infection outbreak is a challenge in responding to community illnesses. A tool that enables anticipation of an increasing bioburden from the community to the particular healthcare facility can be valuable and beneficial to improving patient outcomes. Syndromic surveillance tools have historically not always proved useful at the facility level because they might not help make timely decisions. Forecasting technology can be valuable for infection prevention and useful to combine syndromic surveillance technology with historical data specific to a healthcare facility.

The infection prediction platform 110 includes features to generate infection data for healthcare system 100, including a redesigned Patient Record and direct submission of antimicrobial use (AU) or resistance (AR) data to the National Healthcare Safety Network (NHSN).

The healthcare system 100 generates and controls an interface on user device 102 to display a patient record and a patient form of form fields that include visual elements to display and collect context specific infection data. The form of visual elements provides an alternative way to control the display and collection of healthcare and patient data. The form of visual elements provides an alternative way to display form fields and collect field values that represent healthcare and patient data. The form fields may be extended to be able to capture various information associated with contextual events that occur in relation to syndrome based surveillance. For example, in relation to an intervention that has taken place, among others.

The healthcare system 100 generates and manages patient records that can surface clinically-relevant patient information from hospital feeds like the electronic health records, pharmacy and lab results, and presents a patient record on an interface of user device 102 in an easy-to-read format. This patient-centered view helps healthcare staff and infectious disease pharmacists make faster decisions with real-time, comparative data. This helps them track patients through the care continuum and investigate cases quickly and completely, making interventions such as isolation, protective precautions and antimicrobial stewardship. In addition, hospitals can set normative values and receive real-time alerts when lab results fall outside the acceptable range.

The healthcare system 100 can enable direct submission of AUR data to help ensure a healthcare facility remains compliant with meaningful use guidelines. The healthcare system 100 can accelerate the collection and aggregation of mandated antimicrobial utilization and resistance data to the NHSN. The healthcare system 100 can notify or alert user device 102 or administrator device 104 when the file is finished submitting or if there any errors that need fixing.

Forecast data regarding infections are distributed by central reporting agencies (e.g. external system 108) to healthcare system 100, providing information about what can be expected from the upcoming season, including estimates about onset, duration and severity. However, some healthcare facilities can be outliers from trends so more contextual infection data can improve prediction of infections.

Outliers are not uncommon. For example, in a town in the northeastern United States, for example, flu season consistently comes early. It can happen because residents of the town frequently travel internationally, and bring different strains of the bug back with them, causing flu outbreaks earlier than in neighboring towns, for example. For this town, and others across North America who experience similar trends, context based data is important. Without it, they lose their competitive edge on the flu. In another example scenario, a food festival may be taking place in proximity to several hospitals.

An outbreak of stomach flu (as symptomatically tracked) at these hospitals may be a regular occurrence. On the other hand, an outbreak of stomach flu at a further hospital may not be a regular occurrence and may need to be flagged. Finally, there may be differences as between wards and/or sections of a hospital itself (e.g., a fungus that typically shows up in relation to compromised immune systems starts causing symptoms in a strong, healthy population).

The same is true for other pathogens and communicable diseases. While Healthcare Personnel Infection Prevention or Infection Preventionist (IP) experts can help in mitigating risk and responding to outbreaks, they are often working with information that does not account for their community's unique bioburden (e.g. context data). Even with the best centralized information, this loss of context can limit IPs' ability to proactively respond to potential outbreaks within their healthcare institution's community.

Some of the information that has value for IPs is the product of syndromic surveillance, which refers to methods relying on detection of clinical case features that are discernable before confirmed diagnoses are made. Syndromic surveillance can act as an early warning system to signal potential outbreaks. Syndromic surveillance can be a valuable tool by aggregating a wide range of information from average temperature to subjective judgements about patient symptoms using infection prediction platform 110.

Syndromic surveillance can be conducted by a central agency (e.g. external system 108), which aggregates data from across an area, interprets it and sends the interpretations back to individual healthcare institutions via healthcare system 100. Individual nuances can be lost in this process and time delays in returning the data can make the difference between catching an emerging threat just in time or a little too late.

The infection prediction platform 110 can provide infection prevention teams with the ability to combine the proven effectiveness of syndromic surveillance with their individual, community based data managed by healthcare system 100. The infection prediction platform 110 can enables CBSS. The infection prediction platform 110 can leverage automated analytical techniques to help address a challenge infection prevention teams face: knowing when to respond to infections in a strategic manner. The infection prediction platform 110 can continuously collect, analyze and interpret data specific to a healthcare institution and the population it serves.

The infection prediction platform 110 provides IPs (operating user device 102) with an earlier way to recognize the unique community bioburden that appears on their doorstep on an ongoing basis, but within the context of a specific healthcare facility or hospital. The infection prediction platform 110 can allow institutions to not only monitor changes to their bioburden in real-time, but can combine present and historical data to help forecast the potential signal of a significant event, warning further investigation or intervention. The infection prediction platform 110 generates alerts in response to such forecast or detection. Feedback on the alert classification can be received from user device 102 to refine the prediction model and rules. The infection prediction platform 110 can support IPs in their efforts to move from reactive to proactive and provide additional information to inform responses. The infection prediction platform 110 can help stop a disease as it begins its journey through a healthcare facility, instead of allowing it to proliferate.

The infection prediction platform 110 can be a response to changing realities in infection prevention. Bacteria resistant to the carbapenem class of antibiotics, which are considered the “last resort,” are becoming an urgent threat. Globalization means outbreaks like Zika and MERS-CoV are spreading with greater ease and are increasingly difficult to track and control.

Within hospitals, healthcare-associated infections (HAIs) continue to pose a risk. At the institutional level, an emphasis on quality metrics and incentives and penalties can challenge infection prevention, quality and risk teams to work closely together. Increased regulatory attention is being focused to further drive the risks of HAIs down with limits to reimbursements for associated care of certain HAI's. Prevention of Ventilator Associated Pneumonias (VAPS), Central Line Blood Stream Infections (CLABSI's), Surgical Site Infections (SSI's), Catheter Associated Urinary Tract Infections (CAUTI's), Health Care Provider Vaccination's (HCP) CDI rates and Multi Drug Resistant Organisms (MDRO's) are required to be reported to NHSN through the CMS.

Combined, these changes mean that there is a need to recognize and control pathogens and communicable diseases early. The infection prediction platform 110 can have forecasting capacity to allow IPs to get ahead of the curve to interrupt the chain of transmission and prevent healthcare acquired infections within their institution. The infection prediction platform 110 can provide decision support for booking extra staff, adjusting patient flow, purchasing personal protective equipment or medication, implementing isolation procedures, and so on. Institution-based forecasting abilities can allow IPs to respond judiciously, which can lead to financial and resource savings for organizations and decreased risk of infection and better outcomes for staff and patients.

Infection control is constantly in flux. Whether it is adapting to new emphases on quality and corresponding incentives, responding to increased public attention on healthcare acquired infections (HAIs) or reacting to emerging outbreaks, flexibility is a component of infection control and prevention. The limit of IPs flexibility is challenged. Growing rates of antimicrobial resistance have experts predicting the onset of a post-antibiotic era, for example. In the past year, Zika, Ebola and MERS-CoV are just a few of the outbreaks that have made headlines. Globalization means that conditions are spreading with greater ease and are increasingly difficult to track and control. The infection prevention teams on the front lines of these challenges can be small. For example, hospitals can have one or less full-time IP, or an equivalent role, on staff. IPs spend most of their time on manual surveillance—leaving them with little opportunity to work on innovative prevention and education initiatives to protect staff and patients.

Although daunting, these realities are challenging healthcare organizations to do better, to be proactive instead of reactive and to set impressive benchmarks and meet them. Healthcare facilities can try to improve preparing for emerging infectious diseases, eliminating HAIs and curbing antibiotic resistance. To meet these goals and create meaningful differences in healthcare, IP teams need robust and comprehensive automated surveillance tools to streamline the amount of time they spend aggregating data behind the scenes.

At a high level, surveillance tools to support IPs can decrease re-admission rates and increase reimbursement rates. Moreover, an expanded toolkit that also supports hand hygiene audits, compliance audits and outbreak management can empower IPs with more time and the flexibility to focus on prevention initiatives by providing risk mitigation services to their organizations. This means IPs engage with teams across your organization and implement programs that are essential to the ultimate goal: keeping staff and patients safer.

The infection prediction platform 110 supports both targeted and hospital-wide surveillance models. The infection prediction platform 110 can receive input data from interface at user device 102. The infection prediction platform 110 can push infection data automatically to an interface at user device 102 through alerts. The infection prediction platform 110 can leverage pre-population by connecting healthcare system 100 to other hospital systems to minimize the amount of data-entry. The infection prediction platform 110 can gather data from an organization's admissions discharges and transfers, electronic health record, pharmacy, laboratory, surgical/operating rooms and radiology feeds, for example. The flow of data and information is maintained within the facility's IT network, unless they select to have remote hosting for their data, and in such case infection prediction platform 110 can be a cloud service. The infection prediction platform 110 can report HAI data to CDC and NHSN. The infection prediction platform 110 can also report antimicrobial use (AU) & resistance (AR) data to the NHSN. Alerts can be customized via administrator device 104 or user device 102 and set up to meet an organization's needs. The data can be exported from healthcare system 100 to others, in different formats.

The infection prediction platform 110 is adapted for improved interoperation and inter system compatibility as the infection prediction platform 110 includes an improved database and data structure backend in a first embodiment. Infection prediction platform 110 dynamically incorporates syndromic surveillance data objects in a backend data structure that has a flexible meta-schema allowing a new or modified taxonomy to be adopted or accommodated.

This is an important improvement as applications are able to continue interoperating with the data backend without modifications or retrofits, so costly testing and re-writing of application programming interfaces for legacy enterprise level applications can be avoided. Further, the metabase allows for an expansion of the underlying data structure schema without the need to take the backend data structure offline whenever syndromic data objects need to be incorporated into the backend.

Syndromic data objects are typically generated in response to inputs received by administrator devices 104 or user devices 102, and can be indicative, for example, that an event has taken place (such as a ribfest), an intervention has taken place (such as enhanced hygiene protocols), among others. These syndromic data objects include a number of variables that can be tracked, such as a potential intervention/event type, an positional location (e.g., GPS coordinates of the event, a hospital ward location), among others.

To track information associated with the syndromic data objects, the syndromic data objects are inserted into the backend data structure as new columns that expand a schema. These columns, in some embodiments, are populated dynamically with field values as information is received. For example, a new column directed to a “Ribfest event marker” may include a positional coordinate distance or temporal distance from the Ribfest event for a healthcare facility that received a health record indicative of a failed stool specimen. Relevant existing health records of failed stool specimen are thus pre-populated for ease of analysis, and new health records of failed stool specimens can automatically be updated as they are entered. The new columns thus expand the schema in a metabase that has special configurations to maintain the integrity of the overall information healthcare information system by using meta-relations, meta-tables, and meta-relationships to manage the new information as cross-referenced foreign keys and parent-child relationships.

The metabase field values can be prepopulated in some embodiments, for example, in a preferred embodiment, pre-populating information such as facility distance from the Ribfest event (e.g., St. Michael's hospital is 0.5 km away, while St. Joseph's hospital is 3.2 km away, and populating this information automatically based on GPS coordinate data provided). For intra-ward distances, an adjacency matrix may be used, for example, mapping out distances or connections between specific wards. The adjacency matrix can be a look up table (e.g., a 0 shows that the two wards are connected, a 1 shows that the two wards have a ward in between, etc.).

In some embodiments, after an event is completed and the surveillance of the event and its impacts is no longer necessary, the backend data structure is shrunk by removing the corresponding columns. A metabase can be memory intensive, especially as new events are being tracked through expansion of the schema, and thus a corresponding shrinkage of the schema is useful to ensure that memory is efficiently allocated and utilized in the backend data structure.

The healthcare system 100 includes a user interface component on a connected device configured to receive a request to create a new syndromic surveillance data object at a coordinate position of a graphical interface being rendered on a display on the administrator device 104. The new syndromic surveillance data object can include at least a field name and a data value extracted from a corresponding point along an axis of a graph being rendered on the display.

The healthcare system 100 connects to user device 102 and administrator device 104 in various ways including directly coupled and indirectly coupled via the network 106. Network 106 (or multiple networks) is capable of carrying data and can involve wired connections, wireless connections, or a combination thereof. A network 106 may involve different network communication technologies, standards and protocols, including various message buses. The healthcare system 100 connects to external system 108 to exchange data and commands, for example. The healthcare system 100 can also connect to external system 108 via network 106. The healthcare system of some embodiments 100 includes a storage device having a metabase associated therewith to maintain the meta-schema; and at least one processor configured to execute instructions to provide an object insertion engine and a system interface.

The healthcare system 100 includes at least one processor, memory, at least one I/O interface, and at least one network interface. The processor may be a microprocessor or microcontroller, a digital signal processing (DSP) processor, an integrated circuit, a field programmable gate array (FPGA), a reconfigurable processor.

Memory may include computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM).

Each I/O interface enables healthcare system 100 to interconnect with one or more input devices, such as a keyboard, mouse, camera, touch screen and a microphone, or with one or more output devices such as a display screen and a speaker. Each network interface enables healthcare system 100 to communicate with other components, to exchange data with other components, to access and connect to network resources, to serve applications, and perform other computing applications by connecting to a network (or multiple networks) capable of carrying data.

The healthcare system 100 is operable to register and authenticate user device 102 and administrator device 104 (using a login, unique identifier, and password for example) prior to providing access to applications, a local network, and network resources, for example.

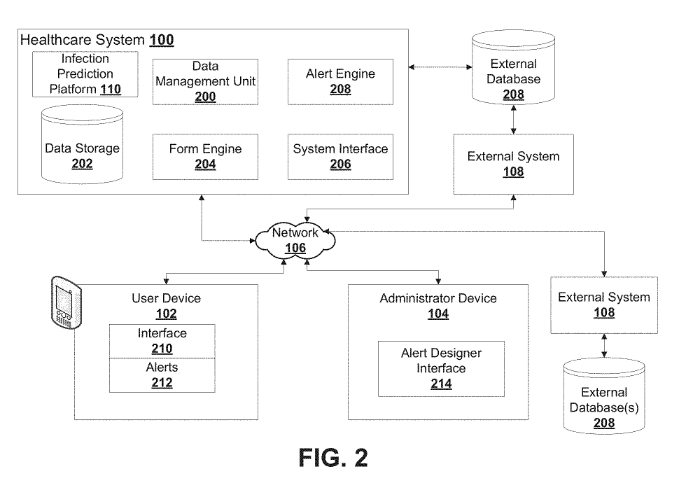

FIG. 2 is a schematic of an example healthcare system 100 according to some embodiments. Healthcare system 100 includes data storage, data management unit, form engine, alert engine, and system interface 206. The user device 102 includes a form interface 210 and alert interface 212. The administrator device 104 includes an alert designer interface 214 to generate rules for infection detection and infection alerts.

Data storage 202 stores infection and healthcare data, alert and pattern rules, visual elements, form objects, form fields, field values and other data.

Data management unit 200 processes rules to evaluate pattern triggers and execute alert actions. The actions can relate to retrieving or generating visual elements for form interface 210. These visual elements can include syndromic surveillance data objects. Types of surveillance data objects include events under investigation, known events, interventions, and “sentinel events”. Sentinel events in particular are triggering logical conditions whose conditions are triggered when an abnormal condition is detected.

In some embodiments, sentinel events include additional logic to exclude contributions from known events that would otherwise skew results (e.g., Ribfest on Monday typically leads to a normal increase in failed stool specimens). In this case, the sentinel event is adapted not to be triggered by numbers of illness events that are closely related to an event tracked by a syndromic surveillance data object maintained within the expanded schema (e.g., a close relationship being computationally determined by a spatial proximity or a temporal proximity to the Ribfest event).

In particular, data management unit 200, responsive to inputs from the administrator device 104 includes an object insertion engine. The data management unit 200 establishes a communication link between the system interface and the user interface component over a network and receives, at the system interface from the user interface component a request to generate the new syndromic surveillance data object.

The object insertion engine is configured to add syndromic surveillance data objects into the backend data structure by expanding the schema. In some embodiments, the administrator device 104 also includes an object removal engine, which conversely removes syndromic surveillance data objects from the backend data structure by shrinking the schema. The data management unit 200 controls the object insertion engine to automatically maintain the electronic representations by establishing one or more new columns corresponding to the new syndromic surveillance data object.

Accordingly, the new syndromic surveillance data object is thus added to a dictionary of field objects in the metabase, wherein an instance of a field object is a form field, and wherein the form field is configured to receive a field value, the metabase structuring the dictionary of field objects into corresponding meta-tables and establishing meta-relations defining parent-child relationships between nodes representing the one or more field objects of the metabase.

The meta-relations are maintained as corresponding foreign keys used to reference the corresponding field objects and the corresponding meta-tables, and the metabase is configured to have a dynamic number of columns that is dynamic according to a number of field objects in the dictionary. New meta-relations are established by data management unit 200 that defines the one or more additional user-defined field objects as new child nodes to corresponding parent nodes of the one or more additional user-defined field objects.

Accordingly, the expanded schema enables analyses to be conducted where the field objects pertaining to the new syndromic surveillance data object are used to store data that can be utilized to track elements of information in association with the new syndromic surveillance data object, such as whether there is a spatial or temporal relationship, etc. The data tracked in the new columns can be processed to identify potential patterns between the event as represented by new syndromic surveillance data object and other field objects maintained on the metabase. In doing so, the metabase maintains electronic representations between the field objects and the form fields, the one or more additional user-defined field objects representing the new or modified taxonomy.

The form interface 210 and alert interface 212 can provide an interactive graphical interface (IGI) to enable user device 102 to review data relative to historic norms (including benchmark data specific to a given health care facility) but to at the same time interact with the data to control and update the IGI and initiate mitigation strategies using control commands to limit impacts of sentinel events.

The healthcare system 100 has infection prediction platform 110 with infection surveillance software that can assist IPs and antimicrobial stewardship teams with access to software tools to move from reactive to proactive prevention of infection risks. The infection prediction platform 110 has rules to coordinate the care for people that are correlated based on the care pathways and symptoms of infection surveillance and healthcare data. The infection prediction platform 110 is configured to crawl through the metabase in generating predictions or extrapolations thereof. For example, a specific query language can be used to interface with the metabase, which unbeknownst to underlying applications, uses the meta-relationships and meta-relations to traverse the metabase in presenting differing information in accordance with the expanded schema elements to legacy and otherwise unmodified applications.

Case definitions for infections are logical conditions. For example, a case definition could be as follows: *At least two (2) or more respiratory symptoms*: fever (Temp >37.8 C) OR abnormal temperature or chills; new or worsening cough OR shortness of breath; runny nose, congestion or sneezing; sore throat OR difficulty swallowing; malaise; myalgia; loss of appetite; and headache. Healthcare records can include symptomatic information obtained, for example, from laboratory data (e.g., N.P swabs (+ve) Influenza virus—(H1N1) Strain (majority) Sputum (+ve) Influenza virus—(H1N1) Strain Legionella urine antigen test—Neg.).

The infection prediction platform 110 can enable infection control with syndromic surveillance and continuous collection of health related data for a health care facility. The infection prediction platform 110 can generate alerts as an early warning for public health emergencies. The infection prediction platform 110 configure rules for context based alert generation. In the context of the expanded scheme resultant from the insertion of the syndromic surveillance data object, the rules for context based alert generation may be modified based on contextual factors stored on the new columns and corresponding field objects. For example, a proximity to a known event (e.g., Ribfest), may cause a particular failed stool sample that otherwise seems extraordinary to be removed from a data set for alert generation (e.g., Ribfest is the known factor for the increase in failed stool samples, a query of the data value indicates that the new columns stored a proximity distance of a hospital that is very close to where the Ribfest occurred).

The infection prediction platform 110 can use the existing mapping system of health care data of health care system 100. The infection prediction platform 110 can configure rules for a hospital, for example, and provide location specific context (e.g. demographic). Location specific context can include a determined distance to an event. This distance can be automatically populated based on healthcare information (e.g., distance between a healthcare facility receiving the patient and where an event took place), among others. In some embodiments, the distance is re-weighted based on a transmission factor that can be established in accordance with various logical conditions. For example, there may be a higher rate of spread due to the impact of population density, transportation links, among others.

The infection prediction platform 110 can apply historical background rate of the hospital and create a trend line on alert interface, for example. A trend may be that one day each year there is an uptick of vomiting and diarrhea symptoms, which may be due to a local food event, for example. The trend event can be correlation with and other context data to detect irregularities (as opposed to “normal” based on historical norms). The infection prediction platform 110 is configured in some embodiments to track a trend line based on a statistical analysis of prior trend events in accordance with the one or more curve fitting techniques such as a rolling average, Holt Winters curve fitting, and polynomial regression, among others. This trend line can include a forecast, in some embodiments, that extends beyond a present time, the forecasting segment determined based on an extrapolation of the trend line.

The infection prediction platform 110 is further configured in some embodiments to include a pattern recognition engine configured to determine a correlation level between a classification of the intervention event and the syndromic surveillance case definition, the correlation level utilized by the trend line determination engine to bias the extrapolation of the trend line, modifying the rendering of the forecasting segment. For example, the extrapolation line could be trended up (in view of an event marker close to the present indicating that there will be an increased incidence due to a known causation event, whose incubation period has yet to fully vest), or trended downwards (in view of an event marker for an intervention event where increased hygiene practices will likely reduce the number of instances). In relation to the forecasting segment, sentinel events may still be triggered when specific conditions are met in the extrapolated data. Accordingly, a visual element corresponding to a sentinel event can still be rendered in relation to extrapolated data in the trend line that extends beyond the present.

The sentinel event automatically “keeps watch” over the raw data, or the trend line, rendering a warning visual indicator when the raw data or the trend line has a characteristic that triggers the sentinel event. This may include, for example, a large amount of unexplained symptomatic cases being present, a forecasted large amount of unexplained symptomatic cases being present, a lack of mean reversion of the trend line over a duration of time, among others. In some embodiments, the sentinel event triggering conditions are adapted only to track unexplained records of symptoms, while in other embodiments, the sentinel event triggering conditions are adapted to track both explained and unexplained records of symptoms.

The sentinel event can be utilized to establish a start duration for an analysis, based for example, on the context of an actual event that has occurred. The sentinel event can be utilized to show that a specific type of event has occurred, and tracks sustained points of interest. The sentinel event is associable with an incubation period duration to account for delayed effects between an event and the emergence of symptoms in the data, and the incubation period duration is tunable depending on a type of infection or the type of sentinel event.

Once staff identify a “sentinel event” depending on the event, a triggered sentinel event may trigger the system to initiate an investigation into the patient records for follow-up. The graphical element for the sentinel event can be interfaced by the user, for example, to add, for example, through a “drop down” interactive visual element, intervention “mitigation” records to select “best practice interventions” and/or leave the record open for further interventions or mitigations.

In some embodiments, a report may be generated along with an action workflow (e.g., saving the report and/or causing an email to be sent). In some embodiments, the alert engine 208 is configured to take automatic actions in the event of a sentinel event being triggered. For example, the sentinel event may track a severe spike in cases that are unexplained. If the spike breaches past a particular threshold, the alert engine 208 is configured in this scenario to raise an urgent alarm, for example, controlling the issuance of a city-wide alert across smartphones automatically without human intervention.

This is especially useful during off-hours of monitoring staff, or during times when a monitoring system cannot be manned by humans. Another scenario where this is useful is where an urgent outbreak must be contained (e.g., Ebola), and instead of sending an urgent alarm, automatic workflows are taken to isolate entire wards of a hospital, or to activate negative pressure machines, among others. In a further embodiment, the sentinel event may be associated with tracked symptoms that may indicate that a disease thought to have been eradicated has returned (e.g., polio, smallpox, measles). In this case, the alert engine 208, upon the sentinel event being triggered, may automatically identify patients or individuals who are not protected by vaccines or otherwise immuno-compromised based on their electronic health records, and broadcast a signal targeted to these individuals for increased vigilance and/or safety precautions (e.g., “SMS Alert→An abnormally large number of symptoms linked of measles have been detected! Please take enhanced airborne/droplet precautions as measles is highly contagious!”).

The alert indicates that there is an event within the community that needs to be investigated and can, for example, be associated with the transmission of a list of actions to be taken. The infection prediction platform 110 can process infection data related to syndromes to detect disease related patterns. The infection data can be tagged with disease or events, for example. The checklist can include precautions and can help hospitals show that they are compliant with best practices. The infection prediction platform 110 can help measure the bio burden of the community to help plan community focused best practices when the service demand hits. Symptoms can represent different diseases and infection prediction platform 110 can detect patterns with additional data parameters. These patterns can be populated into the new columns in the metabase, as events are probabilistically associated with one or more different diseases.

The infection prediction platform 110 can help categorize the symptoms to move the patient to a specific zone or care pathway to minimize impact on the general population. For example, people may come to ER with respiratory illness and related symptoms (air borne) or gastro illnesses (contact based).

The infection prediction platform 110 can integrate with contact tracing system to generate a context pathway for symptoms and events. The infection prediction platform 110 can assign a person who enters a hospital a risk of transmission for tracking. The infection prediction platform 110 can try to isolate patients with undetected illness to mitigate disease spread and proximity to patients with weaker immune system (e.g. high propensity to pick up a disease and a high propensity to spread the disease). The infection prediction platform 110 can implement contact tracing using cell phone identifiers, for example, to track mobility of patients within a geographic location, such as a hospital. Example technical details regarding contact tracing are described in U.S. patent application Ser. No. 15/217,372 titled SYSTEMS AND METHODS FOR NEAR-REAL OR REAL-TIME CONTACT TRACING, the contents of which is incorporated by reference.

The infection prediction platform 110 can add contextual healthcare data for a healthcare facility to syndromic surveillance data.

As an example, the infection prediction platform 110 can tag epidemiologic data with postal code or other geotag and overlay the data on a real-time map to provide a dynamic alert interface 212. The infection prediction platform 110 can generate information reports for different stakeholders.

The form engine 204 interacts with a form interface 210 to render forms having form fields and corresponding form field values to receive and transmit infection and healthcare data.

The form fields include visual elements to receive field values representing infection, healthcare and feedback data. The form engine 204 generates a mapping between the form fields and visual elements. When a visual element is used to receive field values the form engine 204 and the data management unit 200 can use the mapping to link the field values to the form fields in data storage 202. The form interface 210 interacts with system interface 206 to create or update infection and healthcare data in database 202 based on data received using the visual elements. The form engine 204 interacts with a form designer interface 216 to create, customize and configure forms, form fields, and visual elements rendered by the form interface 210. Healthcare system 100 and form engine 204 may integrate features of the healthcare workflow system described in U.S. Provisional Patent Application No. 62/353,707 filed Jun. 23, 2017 this contents of which is hereby incorporated by reference.

The form engine 204 interacts with a client-side component (e.g. form interface 210) to render forms (with form fields for receiving form values) on user device 102. The form may operate in submission mode (the creation of new files) and management modes (the modification of files to add additional form fields and field values, adding follow-up actions to be taken on a file, for example). As an illustrative example, the form engine 204 may display data from a patient file stored in database 202 on user device 102 as a set of form fields represented as visual elements (e.g. as part of form interface 210). User device 102 can submit commands to modify data in the patient file through form fields and visual elements. When rendering a form, the form engine 204 may intercept and execute form rules that are configured by administrator device 104. The form rules can map or link visual elements to form fields. In some embodiments, form rules provide instructions to make sections and fields of a form visible or hidden. The alert engine 208 may intercept and execute form rules that are configured by administrator device 104 through an alert designer interface 214. The form rules can map or link visual elements to form fields. In some embodiments, alert rules provide instructions to detect patterns in the infection and healthcare data. Alert rules may be configured using alert designer 214 and stored in database 202 for access by alert engine 208.

In some embodiments, the alert engine 208 interacts with application programming interfaces (APIs) that provide definitions, constraints and rule parameters, visual elements and the like. The alert engine 208 APIs interpret, parse and send defined alert rules to alert engine 208 and user device 102 to generate alerts with visual elements corresponding to alerts. The form engine 204 is configured to identify a set of rules stored in the persistent data store, each rule having a trigger and an action, the trigger relating to the healthcare data in the persistent data store and the action relating to the visual elements. The form engine 204 is configured to update the form interface to display the visual elements to receive the health care and feedback data.

The alert engine 208 detects events based on received or updated infection and healthcare data. The events may also relate to interactions with the visual elements of the form interface to 10. The alert engine 208 stores event entries for the detected events to an events queue stored in data storage 202. Each rule can have a trigger and an action relating to the healthcare data in the data storage 202, for example.

Healthcare system 100 can have an alert engine 208 to transmit alert notifications to an alert interface 212 on user device 102 based on rules and events. The alerts can also include visual elements as an alternative mechanism to provide notification and alerts as part of an alert interface to 12. Accordingly the alert engine 208 can use rules to link alerts to one or more visual elements to be displayed as part of the alert interface 212.

In some embodiments, healthcare system 100 is configured for dynamically generating a form interface 210 for receiving electronic data relating to a health care incident at a user device 102. The electronic form interface 210 can implement a flexible meta-schema allowing a new or modified taxonomy to be adopted or accommodated without modifications to applications that process the electronic form interface 210. The data management unit 200 and the storage device 202 can implement a metabase associated therewith to maintain the meta-schema. Further details on the meta-schema and the metabase are provided in U.S. application Ser. No. 14/295,637 and U.S. Pat. No. 8,781,852, the contents of which are hereby incorporated by reference. As described herein, the metabase improved in various embodiments to incorporate syndromic surveillance data objects to flexibly extend a schema in response to inputs received at an administrator device 104 indicative of an event to be tracked within the metabase, the metabase providing an adaptive schema that can be extended or shrunk without requiring modifications to underlying applications that utilize the metabase as data storage (e.g., to these applications, the schema appears to remain constant as the metabase utilizes an innovative combination of foreign keys, meta tables, meta relations, and meta relationships to access information stored on new columns representing the extended schema).

Shrinking the schema on storage device 202 is important for memory savings rationales, as the metabase can become unwieldy when overextended, and accordingly, some embodiments include an optimization component that acts as a memory usage “garbage collector” that reclaims memory for re-use by shrinking the expanded schema when the new columns are no longer needed (e.g., responsive to a deletion of an event marker on the administrator device 104).

The optimization component is a daemon process that provides automatic memory management functionality that continually monitors usage of the metabase and the rendered interface to ensure, in some embodiments, that only columns in use (e.g., event markers presently tracked on the administrator device 104 or the interface) are maintained, and any unused columns are re-allocated to avoid memory being used for objects which are no longer in use.

A form engine 204, alert engine 208, system interface 206, alert interface 212, and a form interface 210 establish a communication link between the system 100 and the user device 102 over a network.

The form engine 204 receives from the user device 102 via the network 106, a request to generate the electronic form interface 210 using the form engine 204. The storage device 202 can provide a dictionary of field objects in the metabase. An instance of a field object is a form field, and the form field is configured to receive a field value. The form field can be represented in form interface to 10 as a visual element to receive a corresponding field value. The metabase structuring the dictionary of field objects into corresponding meta-tables and establishing meta-relations defining parent-child relationships between nodes representing the one or more field objects of the metabase. The meta-relations can be maintained as corresponding foreign keys used to reference the corresponding field objects and the corresponding meta-tables. The metabase is configured to have a dynamic number of columns that is dynamic according to a number of field objects in the dictionary. The healthcare system 100 can add one or more additional user-defined field objects in the metabase. The data storage 202 automatically maintains, by the metabase, electronic representations of relationships between the field objects and the form fields, the visual elements, the one or more additional user-defined field objects representing the new or modified taxonomy. The metabase can establish one or more new columns corresponding to the one or more additional user-defined field objects, and can establish new meta-relations defining the one or more additional user-defined field objects as new child nodes to corresponding parent nodes of the one or more additional user-defined field objects.

The form engine 204 can generate the form interface 210 to include an ordered collection of form fields instantiated using the field objects, the ordered collection based at least on the relationships stored in the metabase including at least the meta-tables, the meta-relations, and the new meta-relations, the ordered collection of form fields being a set of visual elements. The form engine 204 can control a display of the user device 102 to automatically return and display, from the system interface 206 to the form interface 210 via the network, the set of visual element. Each visual element can correspond to a potential field value associated with the corresponding field object used to instantiate a corresponding form field. The data storage 202 can define using the metabase a selected visual element from the form interface to 10 via the network, the selected visual element being from the set of visual elements. The form engine 204 can control the form interface to 10 using the set of visual elements based at least on the relationships stored in the metabase to adopt or accommodate the new or modified taxonomy.

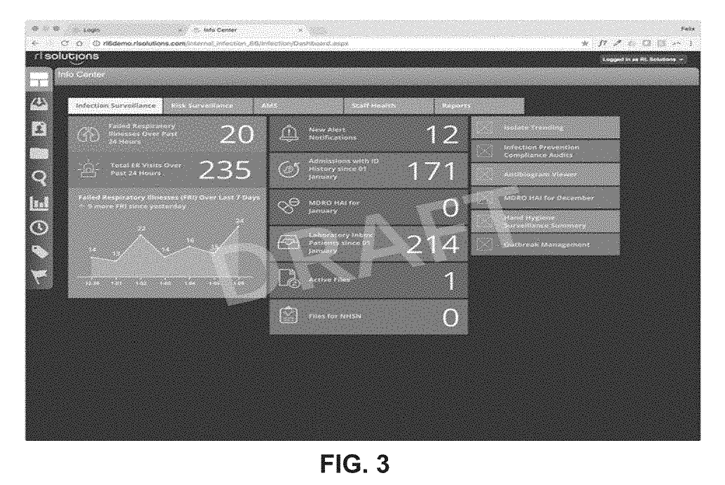

FIG. 3 is an example alert interface with different visual elements for infection surveillance and alerts according to some embodiments. The alert interface includes different visual elements that can trigger control actions such as for example infection surveillance, risk surveillance AMS, staff health, reports, and so on.

Visual items related to infection surveillance can include trend detection for illnesses over a specified time range, events such as ER visits over a specified time, trend reporting for a specified time period, and so on. The alert interface can include visual elements for alert notifications, events for a specified time range, active files, reporting files and so on. The alert interface can also include an intervention and mitigation checklist.

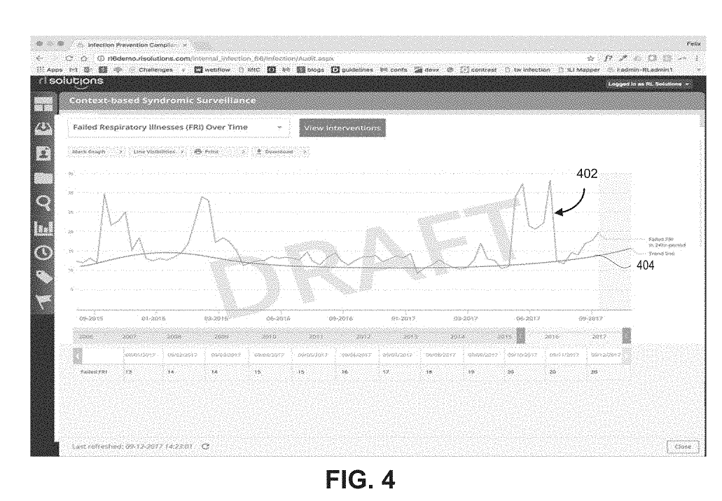

FIG. 4 is an example alert interface with different visual elements for failed respiratory illnesses over time according to some embodiments. The example chart shows a time range of September 2015 to September 2017 the visual elements include events and the number of occurrences for different time periods did the visual element can include a graph 402 with a trend line 404.

During development, Applicants applied used different data sets in order to test the different modelling techniques to be applied for the trend line 404. One such dataset that was tested is the MIMIC™ database which is an openly available dataset developed by MIT Lab™.

By using different data sets and different syndromes, Applicants determined approaches that best minimize the error in forecasting. One such good approach is the Holt Winters model but there are parameters in that model that are needed to be determined in order to minimize the error and these parameters changes for every dataset is provided which makes it harder to use as the parameters may not be consistent. One such logic is to determine the parameters in the backend but is not fast enough for very large datasets.

In some embodiments, a statistical computing data architecture (e.g., such as that provided by the R Project)™ is further added to the system 100 to aid in the determination of the parameters for generating trend lines. In experimental approaches, by integrating the R project to to determine these parameters, a processing speed improvement was found that allowed Holt Winters to become a viable algorithm to use by the ICP's to determine forecasting.

By integrating the R project data architecture, a possibility may be to use other statistical computations in order to better smooth out the curve. In a further embodiment, the statistical computing data architecture is adapted to improve the trend line through an automatic determination of a statistical modelling approach and parameters selected based on a minimization of a forecasting error (e.g., as provided by a mean-squared error aggregate for the points of interest).

Graphs can be created using vector markup language (VML), scalable vector graphics (SVG) or as images (PNG, JPG). An interactive mechanism is provided by infection prediction platform 110.

In another aspect, the graph and the trend line are rendered using the d3™ data visualization tool set, which is more suited for drawing the whole graph, and the one or more interactive graphical event markers are rendered using a React framework which is more suited for creating isolated declarative components, to avoid re-rendering whenever an interaction occurs in relation to a graphical event marker.

Programmatic hooks to events inside the metabase are established such that new elements can be created interactively while the graph is displayed. The problem with using a d3 library only, is that it is based on JQuery which treats the graph as a whole graph and when events are triggered, it renders the entire graph again. This is unacceptable due to the number of interactions by the administrator, slowing down the interface. Optimizations are applied to break down individual elements of the SVG graph to only render objects that caused the events.

These optimizations are applied in relation events, captured as individual elements for rendering. The React™ framework, in particular, is utilized to render the interactive graphical markers after the graph is rendered in accordance with the d3™ framework, improving an overall screen responsiveness and loading time. Data needed by system usually is large, spanning multiple years. By doing lazy loading and applying caching techniques, the data management unit 200 can be configured to speed up the process of data extraction.

The data management unit 200 is configured to build the filter queries and apply different instructions to the command before pulling the data from the data storage backend (e.g., the metabase). This approach is referred to as lazy loading. Results of queries are then stored based on its size and time and can be accessed by a key (e.g., a hash value) if needed. The data used by the data management unit 200 is extracted from the data storage backend, which is a metabase in some embodiments. Other hospital information systems provide packets in the form of structured data sets (e.g., HL7 packets), which store information received from different feeds (admission, lab tests, vital signs, etc.) with different formats. The information is processed, and inserted into the metabase prior to generation of the graphs, trendlines, and other graphical elements thereof.

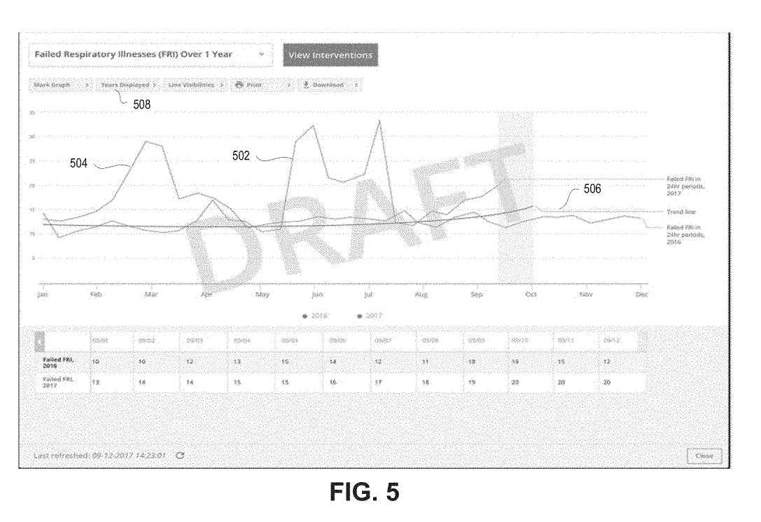

FIG. 5 is an example alert interface with different visual elements to trigger display of failed respiratory illnesses with benchmarking according to some embodiments. For example, the benchmarking may compare failed respiratory illness data over one year time duration against previous years. Different interface-able visual elements are shown at 508, which when selected, shows results for 2017 at 502, results for 2016 at 504, and a trend line 506.

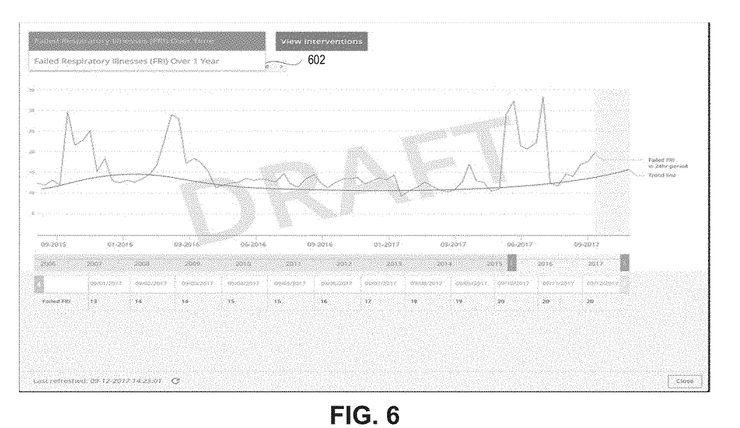

FIG. 6 is an example alert interface with control indicia for different visual elements according to some embodiments. The control indicia 602 can enable alert interface to toggle between different visual elements to show benchmarking data or all available data, for example.

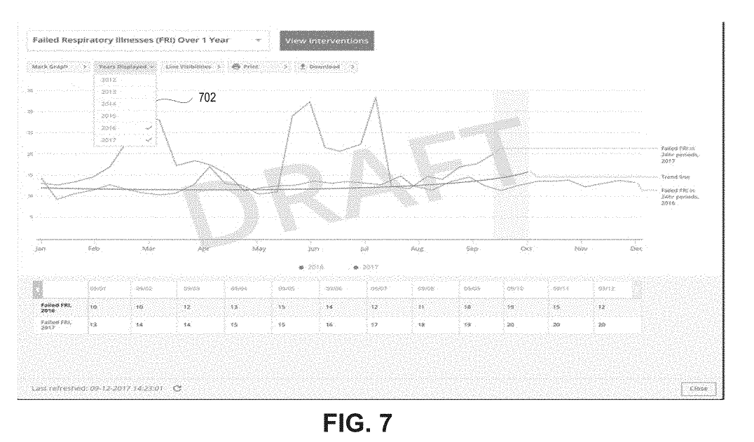

FIG. 7 is an example alert interface with control indicia 702 for different time periods according to some embodiments. The control indicia can enable alert interface to toggle between different years for benchmarking.

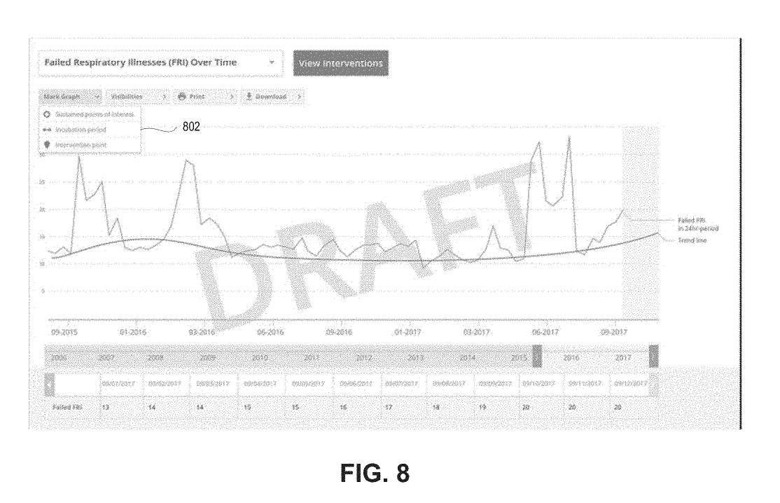

FIG. 8 is an example alert interface with control indicia 802 for different graph displays according to some embodiments. The control indicia can enable alert interface two toggle between markers for sustained points of interest, incubation periods, intervention points, and so on.

When the graph is marked with any one of these event markers, as described in various embodiments, a corresponding backend modification of the metabase can be effected such that the schema is temporarily or permanently expanded to accommodate gathering of additional data set elements as field values in one or more new columns. An event marker may also be removed, and a corresponding backend modification of the metabase can be effected such that the schema is temporarily or permanently reduced to free up memory space.

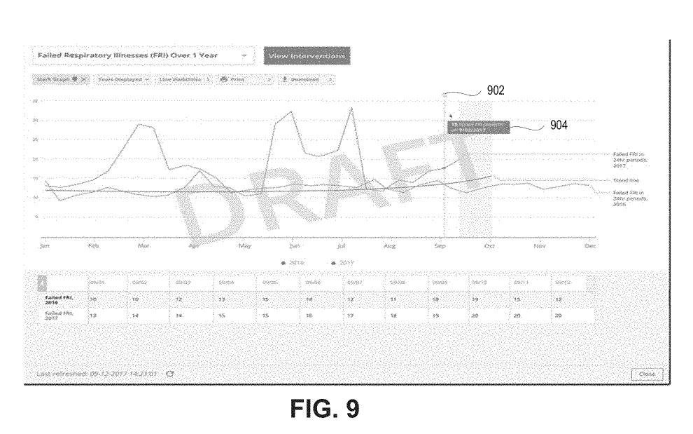

FIG. 9 is an example alert interface with visual elements 902 for different event displays according to some embodiments. The control indicia can enable alert interface to mark graphs of infection data for specific events. The visual element is shown in the form of a balloon visual element 902 with an expanded information bubble 904 showing that there were 18 failed FRI patients at that point on the time axis (2017 Feb. 9).

Other types of visual indicia are possible (e.g., rings, donuts, triangles). In some embodiments, the events are used to indicate events that may explain an increased or decreased set of results (e.g., ribfest leads to more stomach illness symptoms, but that is normal in hospitals proximate to the ribfest, or during Thanksgiving, as many people have left the city for holiday, there may be an expected decrease in the norm).

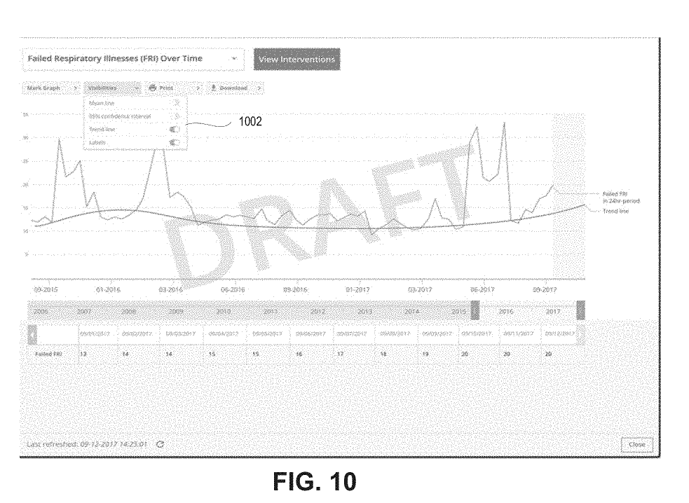

FIG. 10 is an example alert interface with visual elements 1002 for different visibility displays according to some embodiments. Control indicia can enable alert interface to toggle between different visibility characteristics and add marking such as mainline, trend line, labels, confidence intervals, and so. The control indicia can include a print or download option as well. In some embodiments, the system is adapted such that explained events and their associated cases are removed from the data set.

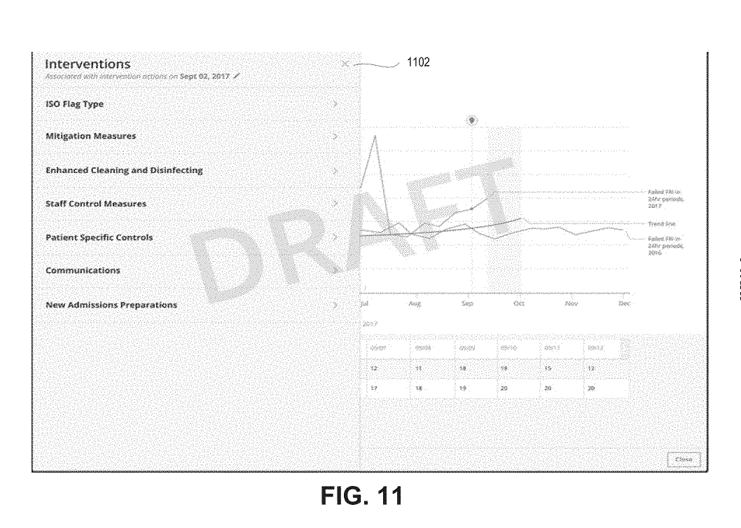

FIG. 11 is an example alert interface with visual elements for different interventions according to some embodiments. The alert interface can include different interventions such as for example ISO flag type, mitigation measures, enhance cleaning and disinfecting, staff control measures, patient specific controls, communications, new admission reparations, and so on. The alert interface can enable selection of different interventions to generate a dynamic and customized checklist report. A sidebar 1102 is shown that can capture information regarding the intervention, which can be used to pre-populate or populate field values in corresponding field objects the metabase backend when the intervention event is created. The intervention can be used to indicate an expected decrease, for example.

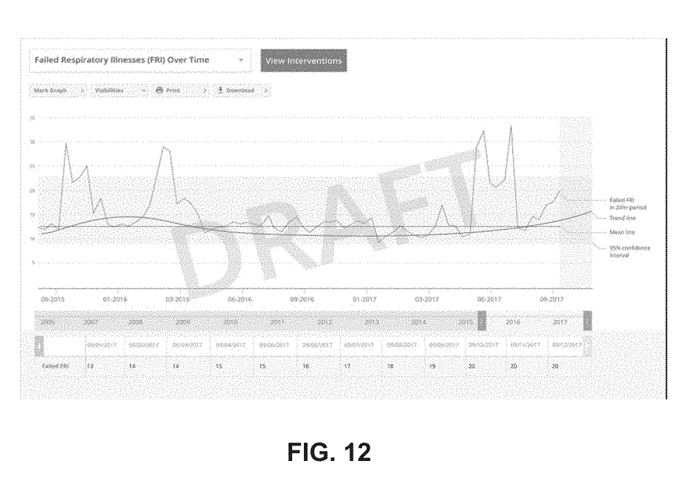

FIG. 12 is an example alert interface with visual elements for interventions over different time periods according to some embodiments. The alert interface shows a graph with a mean line, a confidence interval, and trend line.

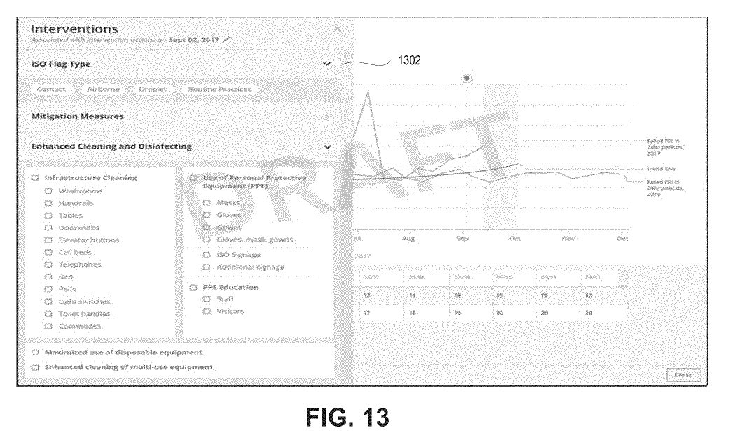

FIG. 13 is an example alert interface with visual elements for a checklist of interventions including cleaning and disinfecting according to some embodiments. Examples include infrastructure cleaning, use of personal protective equipment, education, use of disposable equipment, cleaning of multiuse equipment, and so on. The sidebar 1302 includes a checklist set which correspond to field values of the metabase columns.

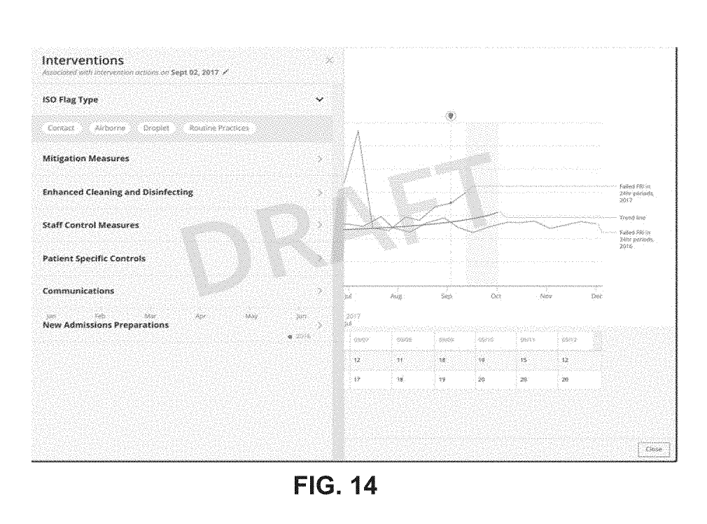

FIG. 14 is an example alert interface with visual elements for a checklist of interventions including ISO flag types according to some embodiments. Example types include contact, airborne, droplet, routine practice, and so on. Similarly, this information can be used to pre-populate or populate field values in corresponding field objects the metabase backend when the intervention event is created. Different types of interventions may have different levels of expected effectiveness depending on the type of symptoms (e.g., glove protocol is useful against contact spreading but less against airborne).

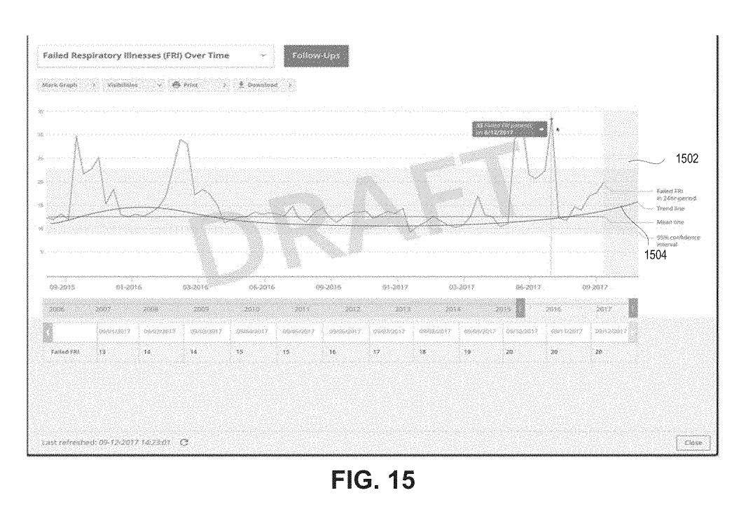

FIG. 15 is an example alert interface with visual elements for interventions including follow-ups according to some embodiments. The alert interface marks different follow-ups on the graph of infection data. A forecast period 1502 is shown. In this example the trend line has an extrapolation segment 1504. In some embodiments, the extrapolation segment 1504 is trended up or downwards depending on various event markers placed proximate to the current date.

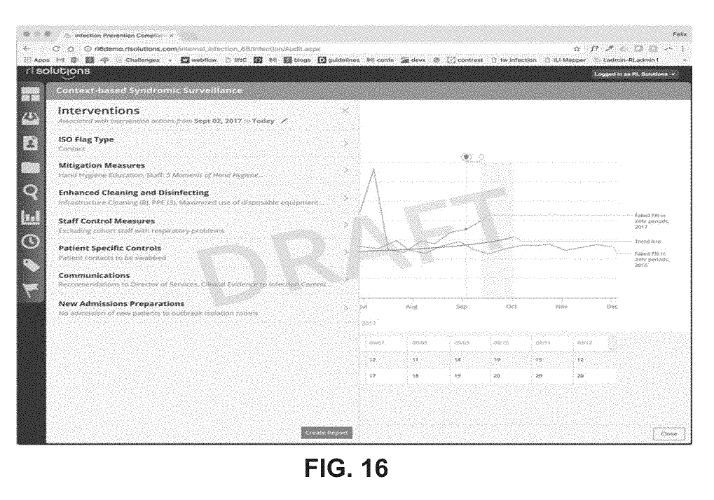

FIG. 16 is an example alert interface with visual elements for intervention controls according to some embodiments. The intervention controls can enable reports and customizations for ISO flag type, mitigation measures, enhance cleaning and disinfecting, staff control measures, patient specific controls, communications, new admissions preparations, and so on.

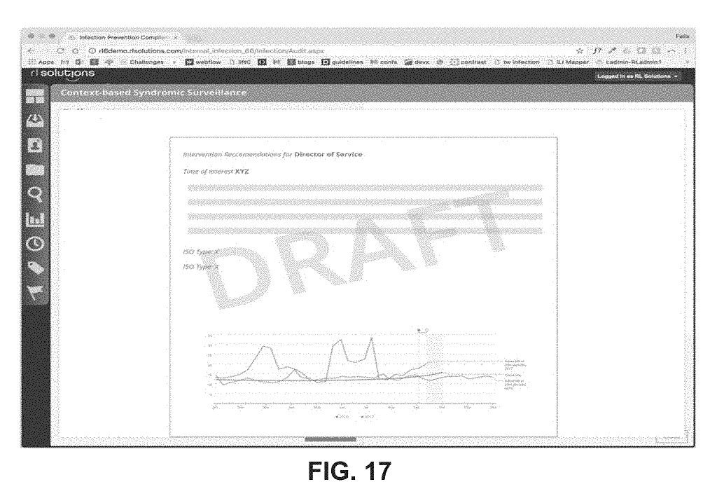

FIG. 17 is an example alert interface with visual elements for an intervention report according to some embodiments. The report can include recommendations for different personnel, times of interest, ISO type, and so on. The report can include recommendations for different mitigation measures. The report can include visual elements for trend data based on the process.

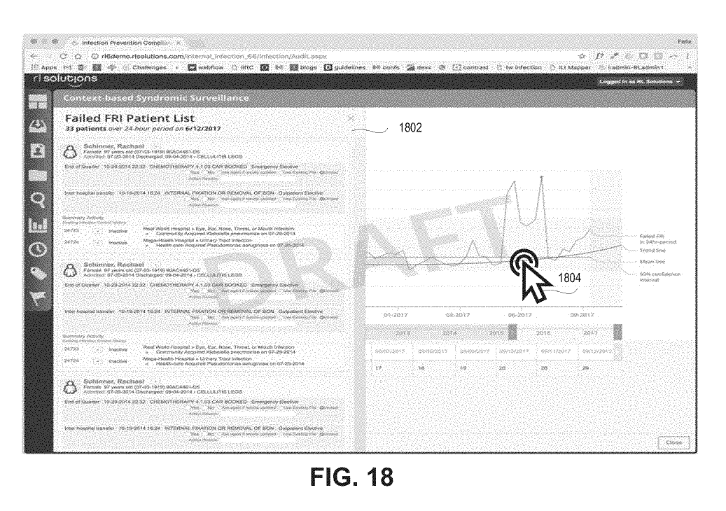

FIG. 18 is an example alert interface with visual elements for a failed patient list according to some embodiments. The list can include different patients related to an event for example. A sidebar 1802 is provided responsive to a mouse cursor position 1804.

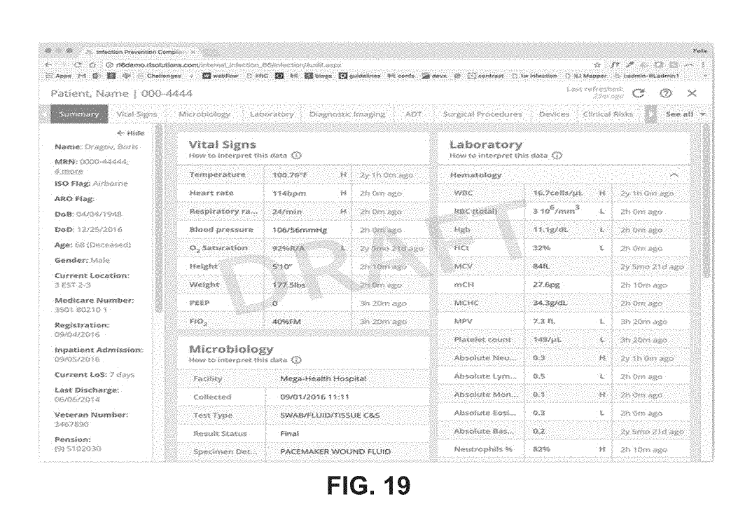

FIG. 19 is an example alert interface with visual elements for triggering generation of different reports according to some embodiments. The reports can relate to vital signs, microbiology, laboratory, and other data related to the patient.

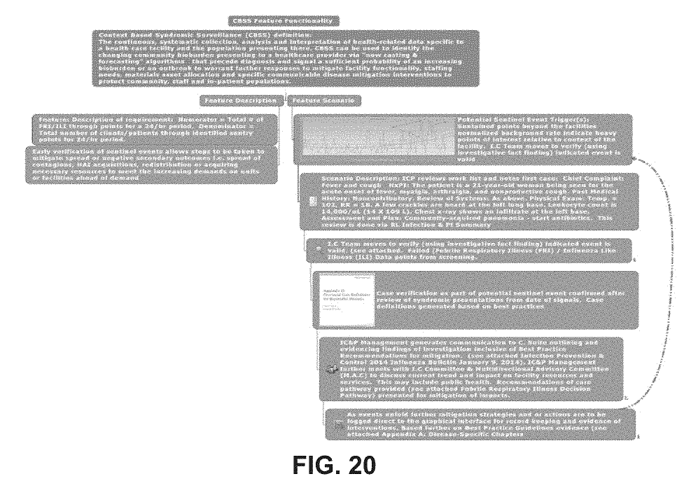

FIG. 20 is an example process for infection surveillance and features according to some embodiments. The process can be used for context-based syndromic surveillance and includes different feature descriptions and feature scenarios the early verification of events allows steps to be taken to mitigate spread or negative secondary outcomes. The early verification of events also enables redistribution or acquiring of necessary resources to meet the increasing demands on units and facilities ahead of the demand. There can be different event triggers. For example event triggers can be sustained data points beyond the facilities normalized background rate to indicate heavy points of interest relative to the context of the facility. The system 100 can then verify that the indicated event is valid by collecting feedback data from user device 102. The system 100 receives symptom data from a form interface for example the symptom data can be correlated with infection data and healthcare data to detect events. As events unfold mitigation strategies and actions can be logged directly to interface for record-keeping and evidence of interventions. The recommendations can include a care pathway for mitigation of impacts.

The form interface can include a screening tool to capture healthcare data specific to a facility or geographic location for example a screening tool can request symptom data, temperature data, respiratory illness contact history, travel associated risk factors, and so on.

An example screening tool question set is provided below for illustrative purposes. From this screening tool, system 100 can collect data point for a scoring system within the decision pathway tree to generate a risk factor metric for a patient. User device 102 can use form to submit data to classify patients as infectious and place within the isolation classification.

### Febrile Illness (FRI) Risk Factor Screening Tool

### Date

### Unit

### Section A: FRI Symptoms

Are you experiencing any of the following symptoms?

New/worse cough (onset within 7 days) YES/NO

New/worse shortness of breath (worse than what is normal for patient) YES/NO

### Section B: Temperature

Is patient feeling feverish, having shakes or chills in the last 24 hours? YES/NO
If yes to symptoms in Sections A or B record temperature

### Temperature Record

Is the temperature above 38° C.? YES/NO

### Section C: Respiratory Illness Contact History

1. Has the pt had contact with a person with (FRI) while not wearing protection against respiratory illnesses in the 10 days prior to onset of their symptoms?

### YES/NO

2. Has the pt been in a healthcare facility, or nursing home in the last 10 days prior to onset of this illness? (insert facility type)

### YES/NO

3. Has Public Health asked pt to remain in home quarantine or isolation in the 10 days prior to onset of this illness?

### YES/NO

4. Have you been to any of the following (identify current outbreak affected facilities in the last 10 days? (insert facility)

### YES/NO

If yes, facility?

### Section D: Travel Associated (FRI) Risk Factors

1. Has the pt, or a member of their household or someone they've had close contact with, traveled within the last 30 days to (name currently high risk country identified for surveillance)?

### YES/NO

If yes, identify country?

### Who?

2. Has the pt been admitted to a hospital* in the 10 days prior to the onset of this illness?
YES/NO If yes, name facility:
3. Does anyone in the pt household, or a close contact, have fever or pneumonia?

### YES/NO

If yes, who?
4. Is the pt a healthcare worker with direct patient contact in a healthcare facility?

### YES/NO

If yes, where?
5. Does the pt live in a nursing home or long term care facility that has had a respiratory infection outbreak in the 10 days prior to the onset of your illness?

### YES/NO

If yes, name facility

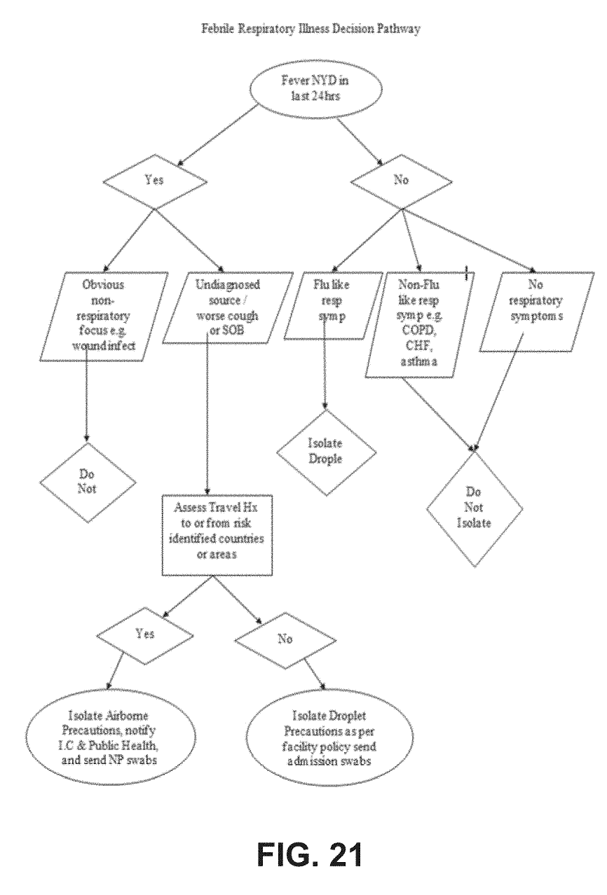

FIG. 21 is an example process for infection surveillance according to some embodiments. An example process can relate to a febrile respiratory illness decision pathway. The process can be used to capture infection and healthcare data. This information may be stored in the metabase healthcare records in field values, which is then accessed when generating a syndromic surveillance graph or visual rendering.

Healthcare system 100 enables users to proactively prevent infections and control infections if they occur. Healthcare system 100 can create interfaces for infection control and prevention, anti-microbial stewardship, outbreak forecasting, hand hygiene audits, contract tracing, staff health, and so on. Healthcare system 100 can customize rules to a particular organization to manage infection prevention and surveillance data. Healthcare system 100 can generate alerts and reports to isolate trending, and customizations for departments, boards and committees. Healthcare system 100 can service one or multiple users. Healthcare system 100 provides a holistic view of an organization's surveillance data.

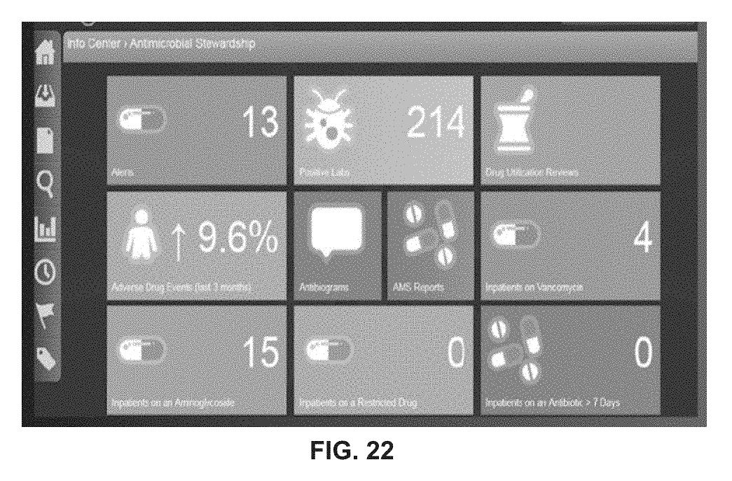

Healthcare system 100 can provide anti-microbial stewardship to ensure the right drugs are reaching the right patients at the right time and for the right duration. Healthcare system 100 can show anti-microbiology use, microbiology and other laboratory results, reports, alerts and ADT information in an interface. Healthcare system 100 can intervene to prevent harm using real-time electronic surveillance to limit drug-related adverse events. Healthcare system 100 can monitor anti-microbiology utilization and can enable custom parameters to track by DDD or DOT. The system can generate utilization and resistance data and report directly to third parties. FIG. 22 is an example interface with different control indicia for report generation.

Healthcare system 100 can provide triage infection evidence to prioritize incoming results that need investigation for potential and confirmed infections. Healthcare system 100 updates confirmed infections using feedback data. Healthcare system 100 can spot trends and monitor and support an organization infection protocol. Healthcare system 100 can track staff health to identify the immune isolation staff need for specific work and track any reactions. Healthcare system 100 can implement instant contract tracing. Contract tracing reports help prevent and mitigate HAIS, MDROS and communicable diseases from spreading. Healthcare system 100 can help forecast outbreaks to interpret data specific to a facility to signal the probability of an outbreak. Healthcare system 100 can connect to sensors to perform hand hygiene audits. There can be a configurable audit tool to survey employees for hand hygiene compliance.

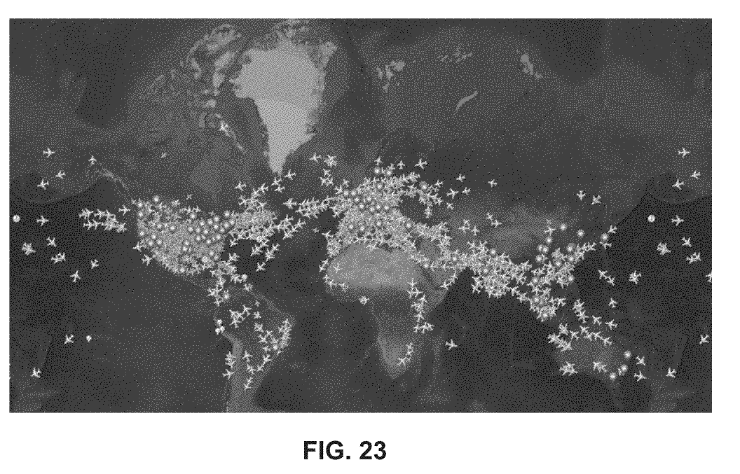

Global travel is prevalent. FIG. 23 depicts an example of travel across the world and example centres of interest. Healthcare Acquired Infections (HAIs), nosocomial infections, outbreaks, contamination, communicable diseases or illnesses, and infectious diseases may originate from the centres of interest. These are all events which no healthcare team wants to face or mitigate within their institutions or care.

However, Risk Management teams and Infection Prevention and Control teams step daily into roles to do just that. People and other health care personnel work to prevent and lessen the impacts of these events while providing ongoing care and service to their communities and employers.

This is against the reality of emerging infectious diseases. These can be diseases which have the real ability to move anywhere potentially, from one place to another in 24 hrs.

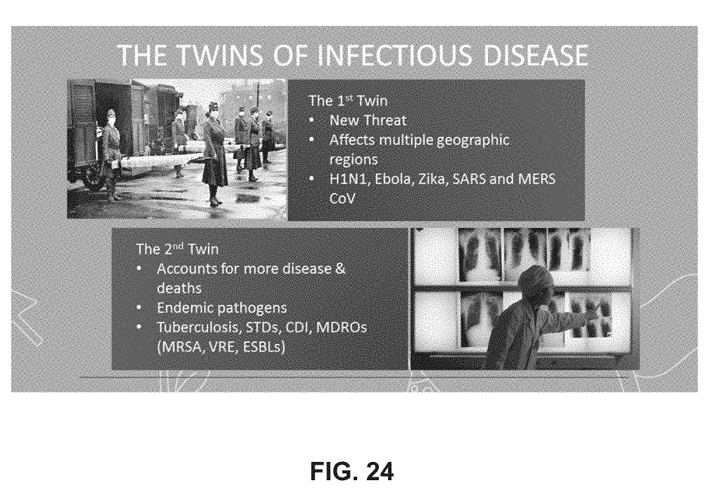

FIG. 24 depicts twins of infectious disease. The first twin—the “new threat” can constantly surprise us. For example, there is the terrifying “Pandemic”, spreading to multiple geographic regions. Pathogens such as pandemic influenza, 1918 Swine Flu (e.g. which may be estimated to have infected ⅓ of the world, causing 50 to 100 million deaths)—H1N1, Ebola and Zika, or previously unknown pathogens such as SARS and MERS are recent occurrences.

The second twin of infectious diseases, is quieter, and less attention grabbing. However, it can account for more human disease and deaths. It is the “Endemic” pathogen. The main categories can be respiratory, skin and gastrointestinal infections. The highest rates of these infections are in the most vulnerable populations, particularly the young and elderly. These are populations especially susceptible to HAI's. There are examples of problematic spread of pathogens.

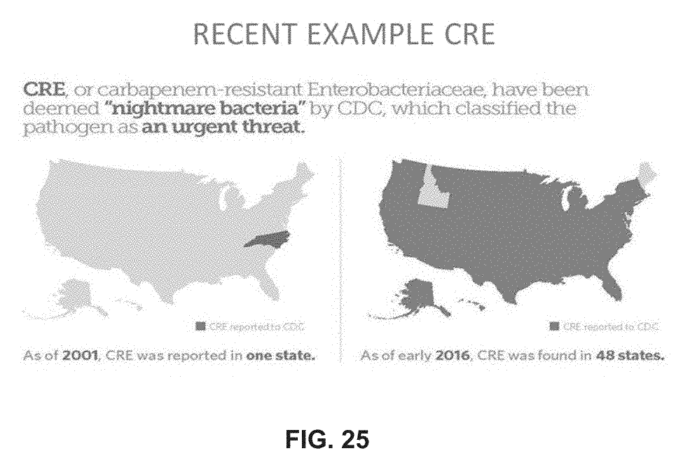

FIG. 25 depicts a recent poignant example, Carbapenem-resistant Enterobacteriaceae (CRE), a family of germs that can be difficult to treat because they can have high levels of resistance to antibiotics. CRE are an important emerging threat to public health. Common Enterobacteriaceae include Klebsiella species and Escherichia coli (E. coli).

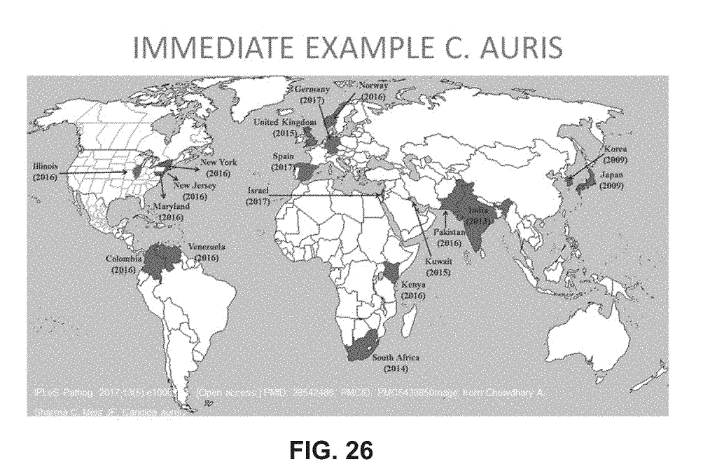

FIG. 26 is an example global map depicting rapid emergence of multidrug-resistant clinical Candida auris strains in 5 continents. The value in parentheses denotes the year of report of C. auris from the respective country or state. From 2009, this was identified first in Japan and Korea. Today, it is now identified throughout the world and the United States. Candida Auris is a multi-drug resistant organism newly identified is spreading faster.

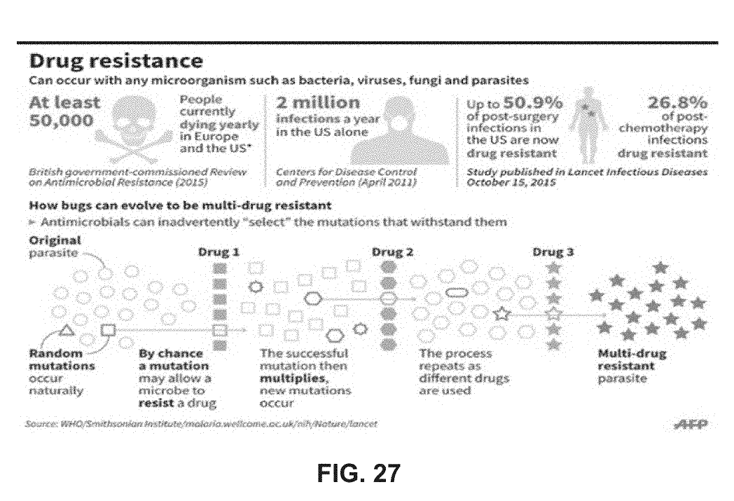

FIG. 27 is an example flow diagram of the development of multi-drug resistant parasites. Antimicrobials can inadvertently “select” for mutations that withstand them. Use of multiple drugs can create multi-drug resistant parasites over time, for example.

The evolving pathogens continue to present ever evolving challenges to healthcare teams. The only constant is that the challenges today will continue to evolve and to challenge the world. Statistics indicate there may be 50,000 drug resistant associated deaths annually, +2,000,000 Infections in the U.S alone, 50% post-surgical infections are drug resistant of some type, and 25% of post chemo therapy infections are drug resistant. This may indicate that the end of the road isn't very far away for antibiotics.

This provides an opportunity for critical synergy between risk management and infection prevention & control. Quality improvements in care and patient safety can require the collaboration and expertise of each service working intimately to share information in the effort to achieve their institutions mandates and objectives.

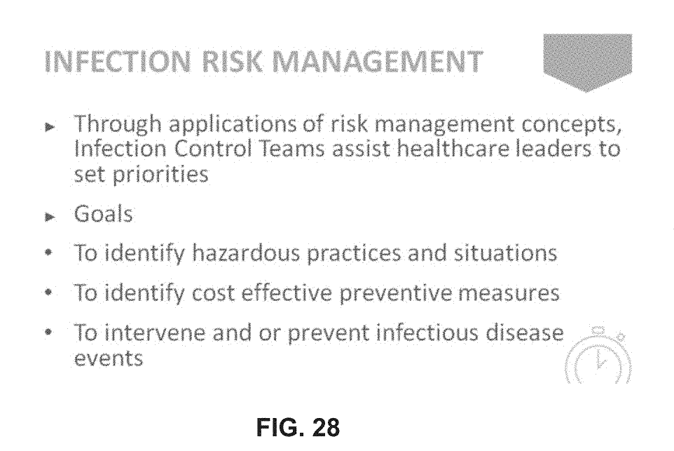

FIG. 28 describes infection risk management. Through application of risk management concepts, Infection Control Teams assist health care leaders to set priorities. Goals can include identifying hazardous practices and situations, identifying cost effective preventive measures, and intervening and/or preventing infectious disease events.

As healthcare settings vary greatly in function, there may not be a universal risk management plan for each. Complexity of care brings many different healthcare providers into the patients care continuum all with a variety of training. Healthcare facilities need to determine their specific risks within their specific context. This can include creating plans to identify their specific risks, unsafe practices and hazards with an aim to assessing severity and frequency. Unexpected death, avoidable HAIs, failure to recognize and treat disease, accidents or equipment failures are just a few examples.

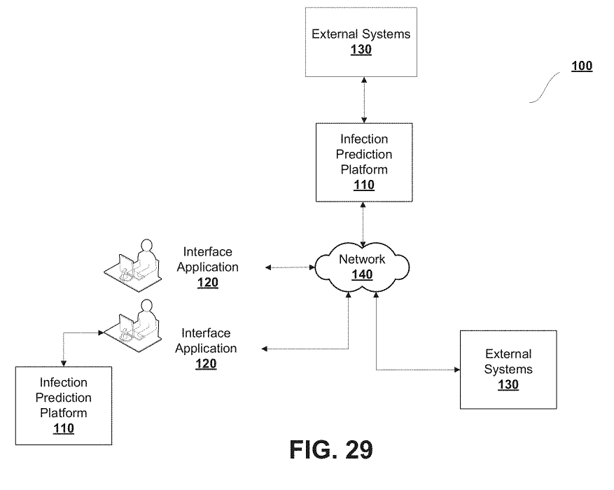

FIG. 29 is an example Context Based Syndromic Surveillance (CBSS) system 100 according to some embodiments that includes Interface Prediction Platform 110. CBSS is the continuous, systematic collection, analysis and interpretation of health-related data specific to a health care facility and the population presenting there.

CBSS system 100 includes infection prediction platform 110. Infection prediction platform 110 connects to interface application 120, for example, to gather data or present data to a user engaged with interface application 120. The data gathered or a modification of the data gathered may encode incidence of Healthcare Acquired Infections (HAIs), nosocomial infections, outbreaks, contamination, communicable diseases or illnesses, infectious diseases, or other health-related data. This may be health-related data specific to a health care facility, for example, where interface application 120 is located. Interface application 120 can sensors to gather data. Infection prediction platform 110 includes a processor specifically configured by logic code stored in memory to implement operations and issue control commands.

Infection prediction platform 110 can receive data from interface application 120, for example, encoding health-related information, for example, an incidence of an HAI. Infection prediction platform 110 can process this data and generate data for the presentation of a display, one or more visual interface elements, for example, a dynamic heat map, one or more interactive interface elements, for example, drag and drop elements, slider bars, and other widgets that enable a user to provide input, for example, dynamic feedback to Infection prediction platform 110 or interface application 120. The interface application 120 can include an Interactive Graphical Interface (IGI) that enables users to review their data relative to their historic norms and at the same time interact with the data and initiate mitigation strategies to limit impacts of sentinel events. The interface application 120 can issue control commands to other components of system to initiate mitigation strategies, for example.

Infection prediction platform 110 can connect to interface application 120 to cause one or more questions to be presented to a user engaged at interface application 120 and to receive one or more responses to questions or other data input from the user. The questions can be presented on a display device using an interface generated by interface application 120. The questions can be presented by way of an audio signal and speaker, as another example. Infection prediction platform 110 can organize the received data or aggregate the data with other data. Infection prediction platform 110 can organize the received data or aggregate the data with other data using time stamps and clock data for synchronization.

Interface application 120 can engage a user, for example, via a display, interactive display, keyboard, mouse, or other sensory apparatus. Interface application 120 can transmit and receive signals or data from such devices and cause data to be sent to Infection prediction platform 110.

In some embodiments, interface application 120 can process data before sending the data via network 140 and/or to CBSS platform 110. In some embodiments, Infection prediction platform 110 connect to interface application 120 over a network 140 (or multiple networks). In some embodiments, Infection prediction platform 110 can connect to interface application 120 directly. Infection prediction platform 110 can receive data from interface applications 120 that are located in different areas in the world. One or more interface applications 120 can be located in the same area or in areas far away from each other.

Infection prediction platform 110 can connect to interface application 120 via a network 140 (or multiple networks). Network 140 (or multiple networks) is capable of carrying data and can involve wired connections, wireless connections, or a combination thereof. Network 140 may involve different network communication technologies, standards and protocols, for example.

In some embodiments, external systems 130 can connect to Infection prediction platform 110, for example, via network 140 (or multiple networks). External systems 130 can be one or more databases or data sources or one or more entities that aggregate or process data. For example, an external system 130 can be a second Infection prediction platform 110.

External systems 130 can receive data from an interface application 120 or Infection prediction platform 110. This data can include raw data collected by interface application 120, such as a single incident or aggregated set of incidents, data processed by interface application 130, Infection prediction platform 110, and/or data from one or more other external systems 130. This connectivity can facilitate the viewing of visual interface elements or displays, manipulation of same, for example, via interactive interface elements, and/or analysis of the data by a researcher, developer, and/or healthcare provider engaged with an external system 130.

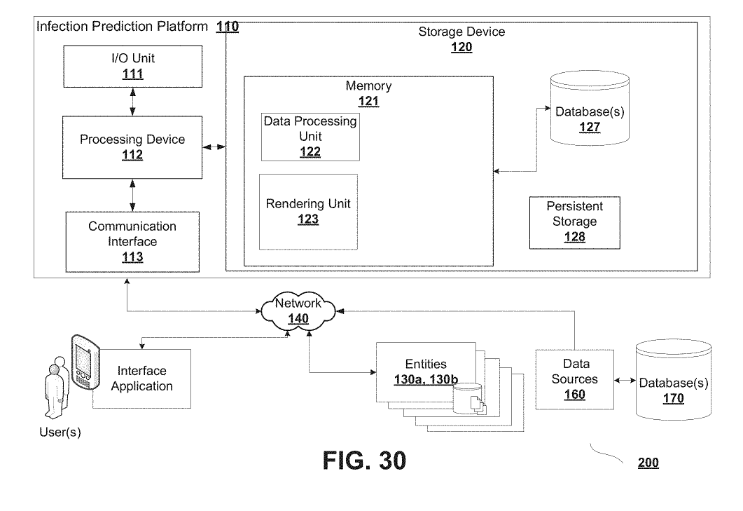

FIG. 30 is a view of an example infection prediction platform 110 and storage device 120 according to some embodiments. Infection prediction platform 110 can include an I/O Unit 111, processing device 112, communication interface 113, and storage device 120.

A infection prediction platform 110 can connect with one or more interface applications 120, entities 130, data sources 160, and/or databases 170. This connection may be over a network 140 (or multiple networks). Infection prediction platform 110 receives and transmits data from one or more of these via I/O unit 111. When data is received, I/O unit 111 transmits the data to processing device 112.

The infection prediction platform 110 controls dynamic rendering syndromic surveillance data objects as interactive graphical visual elements on the display screen. The syndromic surveillance data objects each represent a corresponding event that is being tracked to identify one or more patterns in relation to one or more underlying variables extracted from a backend data structure storing one or more electronic health records.

The communication interface 113 is a data receiver that is configured to extract from the backend data structure on storage device 120 one or more data sets representing healthcare incident records that match one or more logical conditions indicative of a syndromic surveillance case definition.

This can be conducted, for example, by conducting a query executed against a set of field objects stored in the backend data structure. In embodiments where the storage device 120 includes a metabase, the metabase's metaschema is accessed through traversal of the metabase using the foreign key references to access the extended taxonomy of the metabase.

Each I/O unit 111 can enable the infection prediction platform 110 to interconnect with one or more input devices, such as a keyboard, mouse, camera, touch screen and a microphone, and/or with one or more output devices such as a display screen and a speaker. The I/O unit 111 is an input detection component configured to track a signal representing a user input relative to a position on the display screen, in some embodiments (e.g., mouse position).

A processing device 112 can execute instructions in memory 121 to configure classification device 120, and more particularly, data processing unit 122 and rendering unit 123. A processing device 112 can be, for example, any type of general-purpose microprocessor or microcontroller, a digital signal processing (DSP) processor, an integrated circuit, a field programmable gate array (FPGA), a reconfigurable processor, or any combination thereof. The oversampling is optional and in some embodiments there may not be an oversampling unit.

Memory 121 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Storage devices 120 can include memory 121, databases 127, and persistent storage 128.

Each communication interface 113 can enable the Infection prediction platform 110 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. WiMAX), SS7 signaling network, fixed line, local area network, wide area network, and others, including any combination of these.

The infection prediction platform 110 can be operable to register and authenticate users (using a login, unique identifier, and password for example) prior to providing access to applications, a local network, network resources, other networks and network security devices. The platform 110 may serve one user or multiple users.

The storage 127 may be configured to store information associated with or created by the classification device 120. Storage 127 and/or persistent storage 128 may be provided using various types of storage technologies, such as solid state drives, hard disk drives, flash memory, and may be stored in various formats, such as relational databases, non-relational databases, flat files, spreadsheets, extended markup files, etc.

Storage device 120 can be used to create one or more displays, visual interface elements, interactive interface elements, such as drag and drop elements, slider bars, or other widgets allowing engagement with a user. Data processing unit 122 associated with a storage device 120 and Infection prediction platform 110 can receive data, for example, health-related data (such as, incident of disease or spread of disease) via interface application 130. Data collection unit 122 can receive stored data from one or more external systems 130 or interface applications, for example, corresponding to other sessions of data collection. Data collection unit 122 can aggregate or module data to generate data, for example, that encodes spread of disease or pathology across location or across one or more patient characteristics. Data processing unit 122 associated with a storage device 120 can process the data, for example, to remove trends or outliers or errors.

Data processing unit 122 can receive data from single user via interface application 120. In some embodiments, data processing unit 122 can train and generate machine learning classification models using the data received from interface application 120, for example, to predict health-related information, such as location, type, severity, and priority of health-related information, including outbreak of disease, illness, or pathology. Data processing unit 122 can receive stored data from one or more external systems 130 or interface applications 120, such as data corresponding to different locations of interest. Data processing unit 122 can use a classifier to predict spread of disease, illness, or pathology. The result can cause an entity to actuate a response, which can be an alert to a caregiver or data for a researcher via interface application or entities 130. The data can be transmitted for storage at data sources 160 or used at interface application in one or more dynamic visual interface elements, interactive interface elements, or displays, for example.

The rendering unit 123 includes a graphics rendering engine (e.g., implemented on a physical graphics processor) adapted to render a graph along a dimensional axis having a plurality of points. Each point is indicative of a number of the healthcare incident records that match the syndromic surveillance case definition at a corresponding value along the dimensional axis. The rendering unit 123 is configured to render one or more interactive graphical event markers visually proximate to the graph, the interactive graphical event markers each generated responsive to a detected user input. These interactive graphical event markers, when selected for generation by the user, may cause corresponding backend modifications to handle the new event. The new event, as described in various embodiments, is inserted into the backend schema so that the storage 120 is able to add new columns to track field values in field objects in relation to the new event. In generating the graph, data processing unit 122 records one or more associations between the healthcare incident records corresponding to each point of the plurality of points by updating data stored in the corresponding field objects in the backend data structure. These associates are used later for improved speed in providing additional information about the field objects that were tracked as points on the graph (e.g., all cases of failed FRI on January 3).

CBSS rendering unit 123 can process, combine, aggregate, and module data, for example, to generate rendering or interactive components that uncovers and depicts trends or deviation from trends to a user.

The CBSS rendering unit 123 includes a trend line determination engine that is configured to transform the plurality of points into a trend line along the dimensional axis by applying one or more curve fitting techniques to smooth the trend line and to render the trend line as an graphical overlay overlaid in respect of the graph.

These trends or deviations may not be accessible or understood by a user without engaging with the rendered elements or components. For example, a rendered element may be a plot of incident over time for one or more locations, jurisdictions, countries, or areas of interest. In some embodiments, the rendered elements can comprise a heat map depicting incidents or health-related data across locations of interest, using indicia of severity, quantity, incidence, or priority to facilitate easy understanding by a health care provider or user. For example, the indicia can include varied colours each denoting differential severity, incidence, or priority in that location. The rendered elements, for example, the heat map, may include or comprise dynamic components that reflect real-time report of health-related information or disease outbreak at one or more interface applications.

Responsive to the input signal (e.g., mouse position) indicating that the position on the display screen corresponding to the user input is proximate to a point on the graph, the CBSS rendering unit 123, in some embodiments, generates a visual element including visual interface elements corresponding to the set of healthcare incident records corresponding to the point on the graph identified through the recorded one or more associations. This visual element is a pop-up in some embodiments or a side screen widget bar that shows information based on the underlying health data. In a specific example, the pop up or widget bar shows visual elements representative of specific people or records of interactions that show symptoms matching a particular logical definition. In some embodiments, the logical definition is adapted to exclude people whose symptoms are explained by a tracked event. For example, if there is an event established for a ribfest in Scarborough, the additional columns in the metabase may indicate (e.g., through a combination of positional distance and temporal distance) that this symptom may be caused by an expected increase in foodborne illnesses resulting from the ribfest. This specific healthcare record, in some embodiments, is thus excluded from the set of healthcare records.

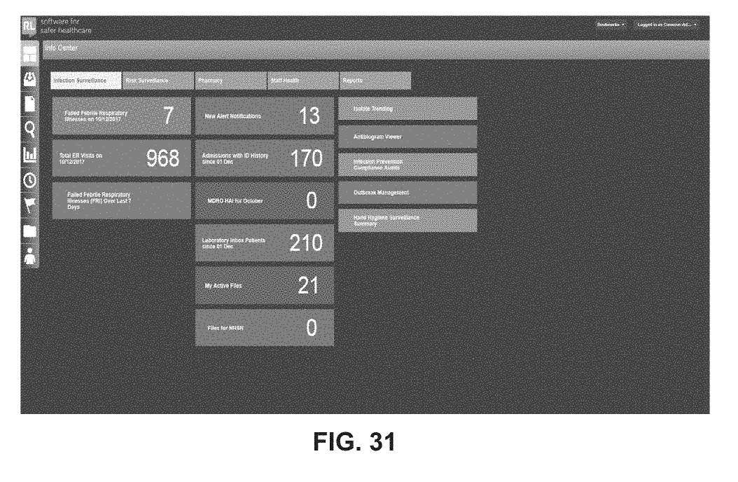

FIG. 31 illustrates an embodiment interface for an Infection prediction platform 110, for example, for identifying and managing health-related data, for example, for infection or other pathologies, including their spread. For example, a visual interface element can be included that displays, in real-time, the total ER visits or failed febrile respiratory illnesses on a given date.

This may facility a new strategy to improve quality outcomes in these areas and may be better designed and implemented through the collaborative work of Risk and Infection Prevention & Control teams. Effective risk mitigation utilizes Infection Prevention & Control audit processes and evaluative analysis to achieve goals through on going measurement and process improvement. Effective infection control practices use a “Risk” lens to understand the more global concerns of health care leaders and communities.

FIG. 31 is a view of an example visual interface element or display generated at infection prediction platform 110. The view is a plot of failed febrile respiratory illness over time, with confidence intervals or ranges. A user can create an indication of an event of interest by placing a marker at a location on the plot, for example, to add an annotation that the data at that point is anomalous.

Infection prediction platform 110 can generate a visual interface element such as a fit line or trend line, for example, depicting mean expected data or mean regression or reversion. Rendering unit 123 can cause an alert or actuate an event in response to a threshold deviation from the fit line or trend line. This can enable alerting to healthcare providers and authorities that unexpected or anomalous disease indicators have been detected by Infection prediction platform 110 and can enable same to identify appropriate action (for example, based on the population, location, or disease type identified by rendering unit 123) and implement action more quickly. Data processing unit 122 can engage a trained classification model to predict the probability of spread or outbreak of a disease or illness.

Infection prediction platform 110 can be used to identify the changing community bioburden presenting to a healthcare provider via “now casting & forecasting” algorithms that precede diagnosis and signal a sufficient probability of an increasing bioburden or an outbreak to warrant further responses to mitigate facility functionality, staffing needs, materials asset allocation and specific communicable disease mitigation interventions to protect community, staff and in-patient populations.

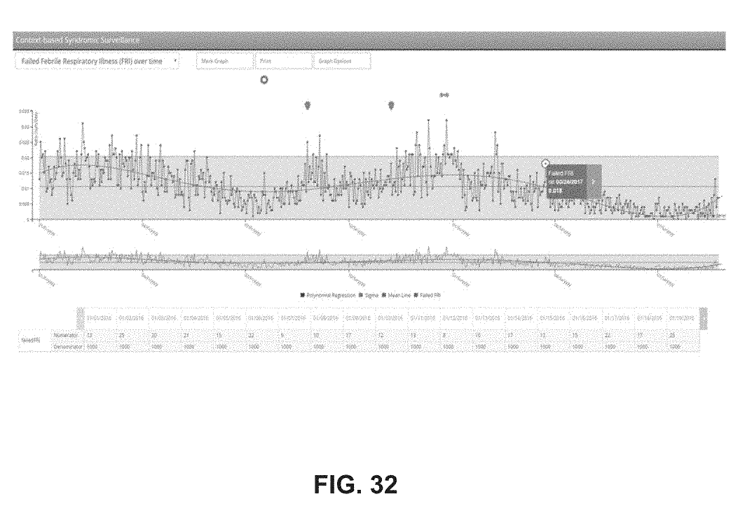

FIG. 32 is a view of an example visual interface element or display generated at infection prediction platform 110 according to some embodiments. A user can interact with the data points, for example, to cause generation of additional visual interface elements or indicate certain response is appropriate.

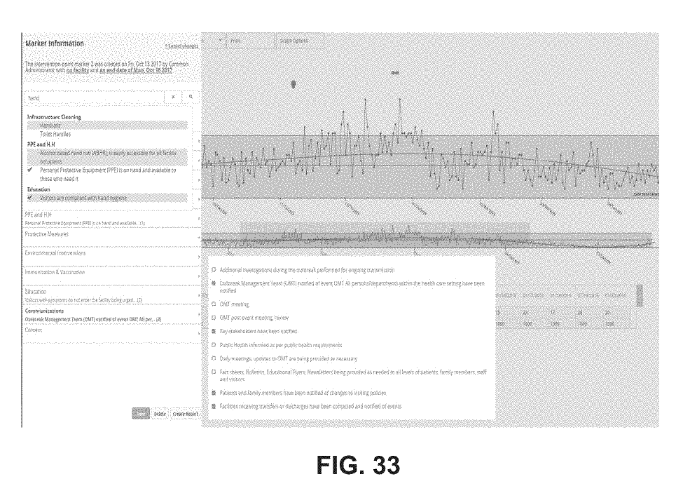

FIG. 33 is a view of an example visual interface element or display generated at Infection prediction platform 110 according to some embodiments. A user can interact with the display to recommend action, for example, to cause correction of the data. This correction can be assessed by or reflected in a visual interface element that depicts deviation from a trend line, mean incidence, or expected data. For example, the action may cause the deviation to be mitigated.

The Interactive Graphical Interface (IGI)—Provides users the opportunity to not only review their data relative to their historic norms but to at the same time interact with the data and initiate mitigation strategies to limit impacts of sentinel events.

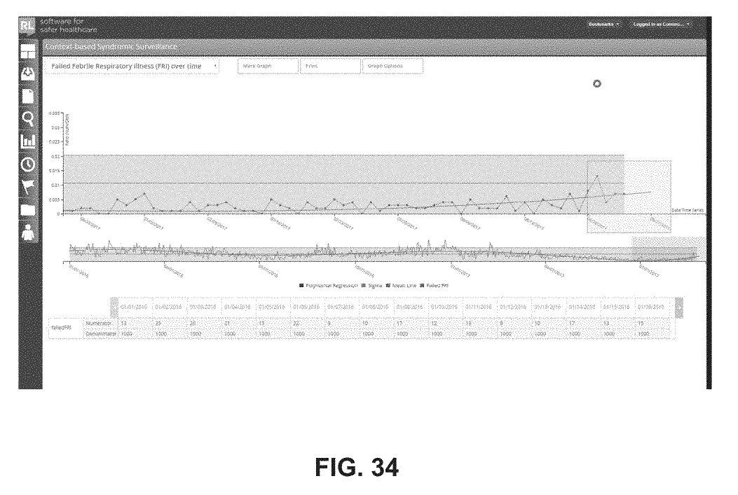

FIG. 34 is a view of an example visual interface element or display generated at Infection prediction platform 110 according to some embodiments. The visual interface element can contain a predictive component for events generated by data processing unit 122 for a future time. This allows a user's own data and a user's own context of the community served to “now cast” and “forecast” trends relative to them through the data analytics platform.

Potential Results can include faster event identification, earlier implementation of Best Practices to break the chain of infection, accurate deployment of staffing and resources, pre-emptive “Risk Avoidance” strategies, improved patient outcomes and satisfaction, better performance of AMS team initiatives, and enhanced protection of those in the care of a user or at their location.

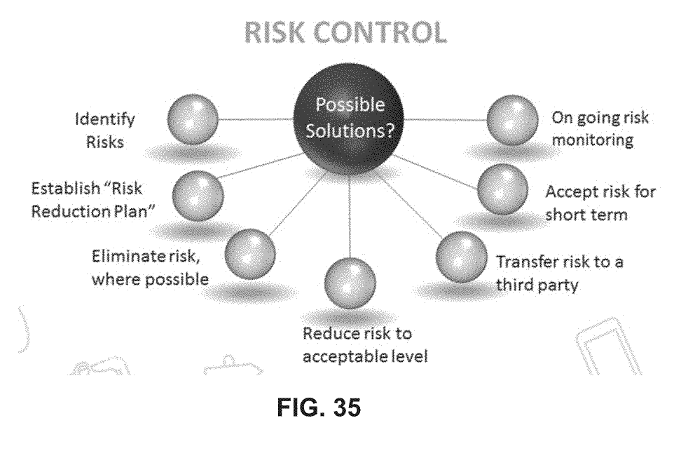

FIG. 35 is a diagram of example risks and mitigation strategies that can be implemented by CBSS system 100. Context Bases Syndromic Surveillance, Antimicrobial Stewardship and Infection Prevention and Control are all necessary in Risk Control and avoidance.

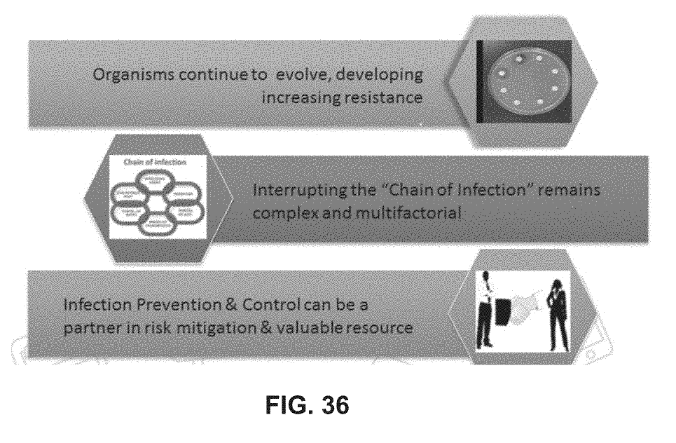

FIG. 36 is a diagram of example context that can be served by CBSS system 100. Pathogens continue to change and resistance continues to challenge care teams everywhere. Communicable diseases and the negative outcomes that come with them need comprehensive management and interventions from teams of care providers. Interconnected Risk and Infection Prevention teams together are uniquely poised to educate and advise executive leaders in their decisions. This can lead to more efficient resource use and better outcomes from those in the care continuum.

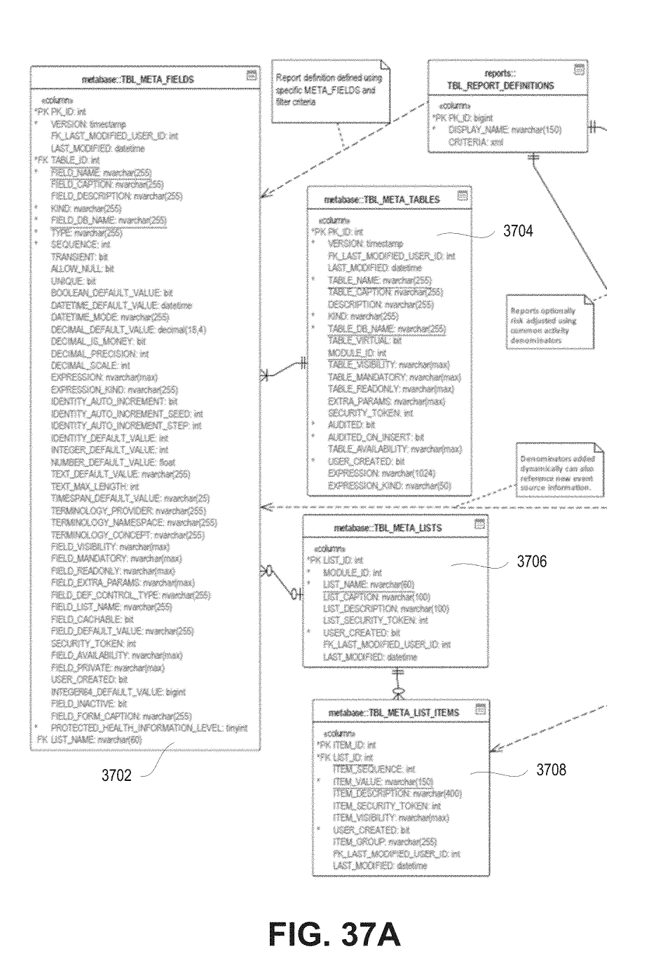

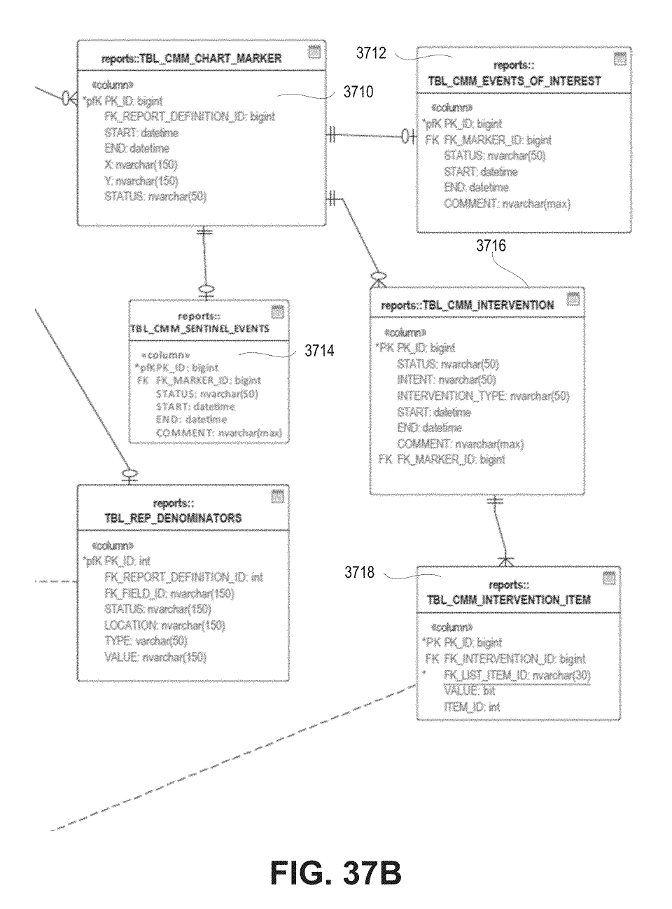

FIG. 37A and FIG. 37B illustrate an example flexible metabase data structure and schema model, according to some embodiments. The flexible metabase structure is an extension of the metabase of U.S. application Ser. No. 14/295,637 and U.S. Pat. No. 8,781,852. In this example, the metabase is a flexible data structure having a metaschema and meta-relationships between meta-fields 3702, meta tables 3704, meta lists 3706, meta list items 3708, event marker objects 3710, which are extended as events of interest objects 3712, sentinel event objects 3714, and intervention event objects 3716 further extended as specific intervention objects 3718. When a new event marker is requested to be drawn on the graphical user interface, the schema is expanded to add the new columns. The new columns are represented and information associated with new event marker are stored as field objects, and in some embodiments, the dictionary of field objects increases in size. The dictionary of field objects encapsulated in the set of meta fields 3702 thus is expanded to track, in the meta tables 3704 information in the form of meta relations between the meta tables 3704 and the tables tracking event markers 3710. The event markers 3710 may include information such as what points of interest and corresponding point on the trend line/axis are relevant for the event marker (e.g., for rendering the event marker in the correct position on the graphical user interface), and there may be start time, and end time, etc. The event markers 3710 are extended as events of interest objects 3712, sentinel event objects 3714, and intervention event objects 3716 further extended as specific intervention objects 3718, which store additional information depending on the type of event marker added by the user. Comment fields may be used to provide information such as incubation time, types of associated illnesses (e.g., Ribfest increases gastroenteritis symptoms), among others. Specific to interventions, specific intervention activities may be further extended as a separate object table 3718 to capture intervention specific information.

As shown, the meta-relations define parent-child relationships between nodes representing the one or more field objects of the metabase. These parent child relationships, provided in the form of foreign keys are used to reference the corresponding field objects and the corresponding meta-tables. Accordingly, while an application connects to the metabase under the assumption that the schema is fixed (e.g., fixed number of columns), the metabase itself is flexibly configured such that the metabase has a dynamic number of columns that is dynamic according to a number of field objects in the dictionary that are reference-able via the meta relations and the corresponding foreign keys.

FIG. 37A and FIG. 37B show different example objects that can be used to generate visual elements for a user interface component on a connected device. The device can be configured to receive a request to create a new syndromic surveillance data object at a coordinate position of a graphical interface being rendered on a display, for example, which can be defined by one of the example objects shown. The new syndromic surveillance data object can have at least a field name and a data value extracted from or corresponding to a point along an axis of a graph being rendered on the display. The objects can store and link different values. The metabase maintains a meta-schema. An object insertion engine is configured to establish a communication link between the system interface (e.g. communication interface 113) and the user interface component over a network to create new objects, which can be defined by one of the example objects shown. The system interface can receive from the user interface component via the network, the request to generate the new syndromic surveillance data object using the object insertion engine (which can be part of the data processing unit 122 and configured by the processing device 112 executing machine instructions stored in the memory 121). The new syndromic surveillance data object representative of an event that is being tracked to identify potential patterns between the event as represented by new syndromic surveillance data object and other field objects maintained on the metabase.

A dictionary of field objects can be stored in the metabase. An instance of a field object is a form field. The form field is configured to receive a field value. The metabase structures the dictionary of field objects into corresponding meta-tables and establishing meta-relations defining parent-child relationships between nodes representing the one or more field objects of the metabase. That is, the metabase can structure relationships (meta-relations) between the different objects in a hierarchy or graph of nodes. The meta-relations can be maintained as corresponding foreign keys used to reference the corresponding field objects and the corresponding meta-tables. The metabase is configured to have a dynamic number of columns that is dynamic according to a number of field objects in the dictionary. The request from the user interface component can trigger the addition of the new syndromic surveillance data object in the metabase. The metabase maintains electronic representations of relationships between the field objects and the form fields and the one or more additional user-defined field objects representing the new or modified taxonomy. The object insertion engine (controlled by a processing device 112, for example) is configured to automatically maintain the electronic representations by: establishing using the metabase, one or more new columns corresponding to the new syndromic surveillance data object; and establishing new meta-relations defining the one or more additional user-defined field objects as new child nodes to corresponding parent nodes of the one or more additional user-defined field objects. The one or more new columns, the meta-tables, and the new meta-relations defining the one or more additional user-defined field objects as the new child nodes in concert represent the new or modified taxonomy incorporating the inserted new syndromic surveillance data object, the foreign keys being accessible for referencing, by the applications that interconnect with the backend data structure, the one or more new columns associated with the new syndromic surveillance data object.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

One should appreciate that the systems and methods described herein may provide example technical effects and solutions e.g. better memory usage, improved processing, improved bandwidth.

Various example embodiments are described herein. Although each embodiment represents a single combination of inventive elements, all possible combinations of the disclosed elements include the inventive subject matter. Thus if one embodiment comprises elements A, B, and C, and a second embodiment comprises elements B and D, then the inventive subject matter is also considered to include other remaining combinations of A, B, C, or D, even if not explicitly disclosed.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information.

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

Example embodiments are described and there may be variations in other embodiments.

What is claimed is:

1. A computer system for dynamically incorporating syndromic surveillance data objects in a backend data structure having a flexible meta-schema allowing a new or modified taxonomy to be adopted or accommodated without modifications to applications that interconnect with the backend data structure, the system comprising:
a user interface component on a connected device configured to receive a request to create a new syndromic surveillance data object at a coordinate position of a graphical interface being rendered on a display, the new syndromic surveillance data object including at least a field name and a data value extracted from a corresponding point along an axis of a graph being rendered on the display;
a storage device having a metabase associated therewith to maintain the meta-schema; and
at least one processor configured to execute instructions to provide an object insertion engine and a system interface, the object insertion engine configured to:
establish a communication link between the system interface and the user interface component over a network,
receive, at the system interface from the user interface component via the network, the request to generate the new syndromic surveillance data object using the object insertion engine, the new syndromic surveillance data object representative of an event that is being tracked to identify potential patterns between the event as represented by new syndromic surveillance data object and other field objects maintained on the metabase,
store in the storage device, a dictionary of field objects in the metabase, wherein an instance of a field object is a form field, and wherein the form field is configured to receive a field value, the metabase structuring the dictionary of field objects into corresponding meta-tables and establishing meta-relations defining parent-child relationships between nodes representing the one or more field objects of the metabase, the meta-relations being maintained as corresponding foreign keys used to reference the corresponding field objects and the corresponding meta-tables, the metabase configured to have a dynamic number of columns that is dynamic according to a number of field objects in the dictionary,
process the request from the user interface component to add the new syndromic surveillance data object in the metabase,
automatically maintain, by the metabase, electronic representations of relationships between the field objects and the form fields, the one or more additional user-defined field objects representing the new or modified taxonomy, the object insertion engine configured to automatically maintain the electronic representations by:
establishing, by the metabase, one or more new columns corresponding to the new syndromic surveillance data object; and
establishing new meta-relations defining the one or more additional user-defined field objects as new child nodes to corresponding parent nodes of the one or more additional user-defined field objects;
wherein the one or more new columns, the meta-tables, and the new meta-relations defining the one or more additional user-defined field objects as the new child nodes in concert represent the new or modified taxonomy incorporating the inserted new syndromic surveillance data object, the foreign keys being accessible for referencing, by the applications that interconnect with the backend data structure, the one or more new columns associated with the new syndromic surveillance data object.

2. The computer system of claim 1, wherein the new graphical syndromic surveillance data object includes at least a syndromic originator spatial coordinate, and wherein during the establishing of the one or more new columns corresponding to the new graphical syndromic surveillance data object, at least one of the one or more new columns is populated with a set of field value representing a spatial coordinate distance between a healthcare facility associated with a data record and the syndromic originator spatial coordinate.

3. The computer system of claim 2, wherein each spatial coordinate distance is weighted by a corresponding transmission speed factor established between the healthcare facility and the event.

4. The computer system of claim 1, wherein the data value extracted from the corresponding point along an axis of a graph being rendered on the display is representative of an approximate point in time in which the event occurred.

5. The computer system of claim 4, wherein the data value is temporally shifted based on a time-lag factor.

6. The computer system of claim 5, wherein the time-lag factor is determined based at least in reference to a lookup table associating a type of infection associated with the event and an incubation period associated with the type of infection.

7. The computer system of claim 4, wherein the new graphical syndromic surveillance data object includes at least a syndromic originator temporal coordinate, and wherein during the establishing of the one or more new columns corresponding to the new graphical syndromic surveillance data object, at least one of the one or more new columns are populated with a field value representing a temporal distance between healthcare record time stamp entry associated with a data record and the syndromic originator temporal coordinate.

8. The computer system of claim 1, wherein the one or more new columns corresponding to the new syndromic surveillance data object includes a binomial value indicative of whether the event has affected the underlying values stored in the field object.

9. The computer system of claim 8, wherein the at least one processor is further configured to execute instructions to provide a graphical display rendering engine that is configured to render a graphical display indicative of one or more records stored in the metabase, the graphical display rendering at least (i) a graph plotting a number of events that match an infection criteria during a duration of time and (ii) a trend line plotting a statistical average of the number of events that match an infection criteria during the duration of time.

10. The computer system of claim 9, wherein the metabase is queried to modify a visual characteristic associated with the plotting of the one or more records whose corresponding binomial values of the one or more new columns indicate that the event has affected the underlying values stored in the field object.

11. A computer system for dynamically rendering syndromic surveillance data objects as interactive graphical visual elements on a display screen, the syndromic surveillance data objects each representing a corresponding event that is being tracked to identify one or more patterns in relation to one or more underlying variables extracted from a backend data structure storing one or more electronic health records, the system comprising:
a data receiver configured to extract from the backend data structure one or more data sets representing healthcare incident records that match one or more logical conditions indicative of a syndromic surveillance case definition by conducting a query executed against a set of field objects stored in the backend data structure;
a graphics rendering engine configured to render a graph along a dimensional axis having a plurality of points, each point indicative of a number of the healthcare incident records that match the syndromic surveillance case definition at a corresponding value along the dimensional axis and configured to render one or more interactive graphical event markers visually proximate to the graph, the interactive graphical event markers each generated responsive to a detected user input;
the data receiver further configured to record one or more associations between the healthcare incident records corresponding to each point of the plurality of points by updating data stored in the corresponding field objects in the backend data structure;
a trend line determination engine configured to transform the plurality of points into a trend line along the dimensional axis by applying one or more curve fitting techniques to smooth the trend line and to render the trend line as an graphical overlay overlaid in respect of the graph; and
an input detection component configured to track a signal representing a user input relative to a position on the display screen;
the graphics rendering engine further configured to, responsive to the signal indicating that the position on the display screen corresponding to the user input is proximate to a point on the graph, generate a visual element including visual interface elements corresponding to the set of healthcare incident records corresponding to the point on the graph identified through the recorded one or more associations.

12. The computer system of claim 11, wherein the graph and the trend line are rendered using a data visualization tool set, and the one or more interactive graphical event markers are rendered using a codified framework that links the event markers to data objects corresponding to at least one of the healthcare incident records.

13. The computer system of claim 11, wherein the dimensional axis is time, and wherein the trend line includes a forecasting segment that extends beyond a present time, the forecasting segment determined based on an extrapolation of the trend line.

14. The computer system of claim 13, wherein the forecasting segment is modified based on a presence of an intervention event as designated by at least one of the interactive graphical event markers, and the system further comprises a pattern recognition engine configured to determine a correlation level between a classification of the intervention event and the syndromic surveillance case definition, the correlation level utilized by the trend line determination engine to bias the extrapolation of the trend line, modifying the rendering of the forecasting segment.

15. The computer system of claim 11, wherein the one or more curve fitting techniques includes at least one of a rolling average, Holt Winters curve fitting, and polynomial regression.

16. The computer system of claim 11, wherein the data receiver is configured to interconnect with one or more healthcare facilities or one or more segments of healthcare facilities, and the one or more data sets include an approximate location distance value for each of the healthcare incident records determined based on an approximate location value for the corresponding healthcare incident record compared against an syndromic originator spatial coordinate representative of a location of a known syndromic event; and
wherein the graphics rendering engine is configured to render an interactive interface component, which when interfaced with, cause the graph and the trend line to be re-rendered absent points representing healthcare incident records having a sufficient proximity to the known syndromic event based on the approximate location distance value being below a pre-defined threshold.

17. The computer system of claim 11, wherein the backend data structure includes a flexible meta-schema allowing a new or modified taxonomy to be adopted or accommodated without modifications to applications that interconnect with the backend data structure.

18. The computer system of claim 17, wherein the backend data structure includes a flexible meta-schema allowing a new or modified taxonomy to be adopted or accommodated without modifications to applications that interconnect with the backend data structure and the system further comprises a storage device having a metabase associated therewith to maintain the meta-schema.

19. The computer system of claim 18, wherein the metabase includes a dictionary of field objects in the metabase, wherein an instance of a field object is a form field, and wherein the form field is configured to receive a field value, the metabase structuring the dictionary of field objects into corresponding meta-tables and establishing meta-relations defining parent-child relationships between nodes representing the one or more field objects of the metabase, the meta-relations being maintained as corresponding foreign keys used to reference the corresponding field objects and the corresponding meta-tables, the metabase configured to have a dynamic number of columns that is dynamic according to a number of field objects in the dictionary; and
wherein new syndromic surveillance data objects are incorporated into the metabase in one or more new columns corresponding to each new syndromic surveillance data object, establishing new meta-relations defining the one or more additional user-defined field objects as new child nodes to corresponding parent nodes of the one or more additional user-defined field objects;
wherein at least one of the one or more new columns is populated with a set of field value representing the approximate location distance value.

20. A computer readable medium storing machine interpretable instructions for dynamically rendering syndromic surveillance data objects as interactive graphical visual elements on a display screen, the syndromic surveillance data objects each representing a corresponding event that is being tracked to identify one or more patterns in relation to one or more underlying variables extracted from a backend data structure storing one or more electronic health records, the machine interpretable instructions, when executed, causing a processor to perform one or more steps according to a method comprising:
extracting from a backend data structure one or more data sets representing healthcare incident records that match one or more logical conditions indicative of a syndromic surveillance case definition, the one or more data sets include an approximate location distance value for each of the healthcare incident records determined based on an approximate location value for the corresponding healthcare incident record compared against an syndromic originator spatial coordinate representative of a location of a known syndromic event;
rendering a graph along a dimensional axis having a plurality of points each indicative of a number of the healthcare incident records that match the syndromic surveillance case definition at a corresponding value along the dimensional axis;
rendering one or more interactive graphical event markers visually proximate to the graph, the interactive graphical event markers each generated responsive to a detected user input;
transforming the plurality of points into a trend line along the dimensional axis by applying one or more curve fitting techniques to smooth the trend line;
rendering the trend line as an graphical overlay overlaid in respect of the graph; and
rendering an interactive interface component, which when interfaced with, cause the graph and the trend line to be re-rendered absent points representing healthcare incident records having a sufficient proximity to the known syndromic event based on the approximate location distance value being below a pre-defined threshold;
wherein the backend data structure is a metabase that includes a flexible meta-schema allowing a new or modified taxonomy to be adopted or accommodated without modifications to applications that interconnect with the backend data structure.
